SEQUENCE LISTING (3 sequences)

SEQ ID NO: 1 - protein, 35 aa, synthetic construct
SGGGSAWSHPQFEKGGGSGGGSGGSSAWSHPQFEK

SEQ ID NO: 2 - protein, 1273 aa, unidentified
  source 1..1273: note = SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1273 aa, unidentified; identical to SEQ ID NO: 2
  source 1..1273: note = SARS-CoV-2

(19) United States
(12) Patent Application Publication
(10) Pub. No.: US 2025/0134985 A1
(43) Pub. Date: May 1, 2025

(54) MEMBRANE FUSION AND IMMUNE EVASION BY THE SPIKE PROTEIN OF SARS-COV-2 DELTA VARIANT

(71) Applicant: Children's Medical Center Corporation, Boston, MA (US)

(72) Inventor: Bing Chen, Westwood, MA (US)

(73) Assignee: Children's Medical Center Corporation, Boston, MA (US)

(21) Appl. No.: 18/684,129

(22) PCT Filed: Aug. 16, 2022
(86) PCT No.: PCT/US2022/075027

§ 371 Date: Feb. 15, 2024

Related U.S. Application Data
(60) Provisional application No. 63/233,703, filed on Aug. 16, 2021.

(51) Int. Cl. A61K 39/215; C07K 14/005

(52) U.S. Cl. CPC A61K 39/215; C07K 14/005; C12N 2770/20022; C12N 2770/20034

(57) ABSTRACT

Provided herein, in some aspects, are methods for using the receptor-binidng domain (RBD) of the SARS-COV-2 S protein, including the the RBD-1, RBD-2, and/or RBD-3 regions, to identify therapeutics for the treatment of SARS-COV-2, developing a vaccine for the treatment or prevention of SARS-COV-2, and identifying a patient as being in need for a treatment for SARS-COV-2.

### RELATED APPLICATIONS

This Application claims priority under 35 U.S.C. § 119(e) to U.S. Patent Application Ser. No. 63/233,703, filed Aug. 16, 2021, and titled “MEMBRANE FUSION AND IMMUNE EVASION BY THE SPIKE PROTEIN OF SARS-COV-2 DELTA VARIANT,” the contents of which are incorporated herein in its entirety.

### REFERENCE TO AN ELECTRONIC SEQUENCE LISTING

The contents of the electronic sequence listing (C123370223WO00-SEQ-AZW.xml; Size: 6,568 bytes; and Date of Creation: Aug. 16, 2022) is herein incorporated by reference in its entirety.

### BACKGROUND

Severe acute respiratory syndrome coronavirus 2 (SARS-CoV-2) is the causative agent of the COVID-19 pandemic (ref(1)). The strain responsibe for the initial outbreak, Wuhan-Hu-1 (ref(1)), was the basis for first-generation vaccine deveoplment. The Delta variant (ref(2)), first detected in India and also known as lineage B.1.617.2, is a variant of concern (VOC) and outcompeted other previously prevalent variants to become a globally dominant strain within several months. It has been estimated that this variant is about twice as transmissible as Wuhan-Hu-1 (ref(1), ref(3), and ref(4)). Infection by the Delta variant appears to have a shorter incubation period with a viral load about 1,000 times greater (or more) than that by earlier variants (ref(5)), but it may not cause more severe disease (ref(6) and ref(7)). Nevertheless, in some individuals, the Delta variant appears to overcome immunity elicited by the first-generation vaccines designed using the Wuhan-Hu-1 sequence (ref(8)-ref(11)). Another VOC, Gamma (lineage B.1.1.28 or P.1), has been widespread in Brazil and some other countries (ref(12) and ref(13)). A third variant, Kappa (lineage B.1.617.1), also first reported in India, remains a variant of interest (VOI) with only a limited surge in certain areas (ref(14) and ref(15)). These SARS-CoV-2 variants highlight the importance of understanding the molecular mechanisms of the increased transmissibility and immune evasion to facilitate development of intervention strategies, such as therapeutics and vaccines, and well as the need for diagnostics.

### SUMMARY

SARS-CoV-2 is an enveloped positive-stranded RNA virus that enters a host cell by fusion of its lipid bilayer with a membrane of the target cell. The membrane fusion reaction is catalyzed by the virus-encoded trimeric spike(S) protein after binding to the viral receptor angiotensin converting enzyme 2 (ACE2). The S protein is first produced as a single-chain precursor, which is then processed by a host furin-like protease into the receptor-binding fragment S1 and the fusion fragment S2 (FIG. 6; ref(16)). After engaging with ACE2 on the host cell surface, the S protein is cleaved by a second cellular protease TMPRSS2 or by cathepsins B and L (CatB/L) in S2 (S2′ site; FIG. 6; ref(17)), initiating dissociation of S1 and a cascade of refolding events in S2 to drive fusion of the two membranes (ref(18) and ref(19)). S1 contains four domains—NTD (N-terminal domain), RBD (receptor-binding domain), and two CTDs (C-terminal domains), protecting the central helical-bundle structure formed by the prefusion S2. The RBD can adopt either a “down” conformation for a receptor-inaccessible state, or an “up” conformation for a receptor-accessible state (ref(20)). The RBD movement appears to be an important mechanism for the virus to protect its functionally critical receptor binding site from host immune responses (ref (20) and ref21)).

Intensive studies on the S protein have advanced understanding of the SARS-CoV-2 entry process substantially (reviewed in ref(22)-ref(25)). In some embodiments, the full-length S proteins derived from the Delta, Kappa and Gamma variants were characterized and their structures determined by cryogenic electron microscopy (cryo-EM). Comparison of the structure, function, and antigenicity of the Delta S with those of Gamma and Kappa, as well as previously characterized Alpha and Beta (26), provided molecular insights into mechanisms of the heightened transmissibility and enhanced immune evasion of the most contagious form of SARS-CoV-2 since its initial outbreak.

Accordingly, in some embodiments, the present disclosure provides methods for identifying a therapeutic for the treatment of SARS-CoV-2, said method utilizing the receptor-binidng domain (RBD) of the SARS-CoV-2 S protein. In some embodiments, the therapeutic is a small molecule. In some embodiments, the therapeutic is a biologic. In some embodiments, the therapeutic is a protein. In some embodiments, the therapeutic is an antibody. In some embodiments, the therapeutic recognizes or binds to the RBD-1 region of the RBD domain. In some embodiments, the therapeutic recognizes or binds to the RBD-2 region of the RBD domain. In some embodiments, the therapeutic recognizes or binds to the RBD-3 region of the RBD domain. In some embodiments, the present disclosure provides methods for treating a patient suffering from SARS-CoV-2, including the step of administering to the patient a therapeutically effective amount of any of the the therapeutics identified.

In some embodiment, the present disclosure provides methods for developing a vaccine for the treatment or prevention of SARS-CoV-2, said method utilizing the receptor-binidng domain (RBD) of the SARS-CoV-2 S protein. In some embodiments, RBD-1 region of the RBD domain is utilized. In some embodiments, RBD-2 region of the RBD domain is utilized. In some embodiments, RBD-3 region of the RBD domain is utilized. In some embodiments, the present disclosure provides methods for administering to a subject an immunogenically effective amount of the vaccine.

In some embodiment, the present disclosure provides methods for identifying a patient as being in need for a treatment for SARS-CoV-2, said method utilizing the receptor-binidng domain (RBD) of the SARS-CoV-2 S protein. In some embodiments, RBD-1 region of the RBD domain is utilized. In some embodiments, RBD-2 region of the RBD domain is utilized. In some embodiments, RBD-3 region of the RBD domain is utilized.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1C show more efficient membrane fusion by the Delta variant than other variants. FIG. 1A shows the time-course of cell-cell fusion mediated by various full-length S proteins, as indicated, with HEK293 cells with no exogenous ACE2. FIG. 1B shows cell-cell fusion mediated by various full-length S proteins with HEK293 cells transfected with low levels (0-0.25 ng) of ACE2 expression constructs. FIG. 1C shows the time course of infection of HEK293-ACE2 cells by MLV-based pseudotyped viruses using various SARS-CoV-2 variant S constructs containing a CT deletion in a single cycle. Infection was initiated by mixing viruses and target cells, and viruses were washed out at each time point as indicated. The full time-course and concentration series are shown in FIGS. 8A-8D. The experiments were repeated at least three times with independent samples giving similar results.

FIG. 2 shows antigenic properties of the purified full-length SARS-CoV-2 S proteins. Bio-layer interferometry (BLI) analysis of the association of prefusion S trimers derived from the G614 “parent” strain (B.1) and the Gamma (B.1.1.28), Kappa (B.1.617.1) and Delta (B.1.617.2) variants with soluble ACE2 constructs and with a panel of antibodies representing five epitopic regions on the RBD and NTD (see FIG. 9A and ref (32)). For ACE2 binding, purified S proteins were immobilized to AR2G biosensors and dipped into the wells containing ACE2 at various concentrations. For antibody binding, various antibodies were immobilized to AHC biosensors and dipped into the wells containing each purified S protein at different concentrations. Binding kinetics were evaluated using a 1:1 Langmuir model except for dimeric ACE2 and antibody G32B6 targeting the RBD-2, which were analyzed by a bivalent binding model. The sensorgrams, as well as the fits, are shown. Binding constants highlighted by underlines were estimated by steady-state analysis as described in the Methods. RU, response unit. Binding constants are also summarized here and in Table 1. All experiments were repeated at least twice with essentially identical results.

FIGS. 3A-3I show the cryo-EM structures of the full-length SARS-CoV-2 S proteins from the Delta, Kappa and Gamma variants. FIGS. 3A-3C show the structures of the closed prefusion conformation and two one-RBD-up conformations of the Delta S trimer in ribbon diagram with the following protomers shown: NTD, RBD, CTD1, CTD2, S2, the 630 loop, and the FPPR. FIGS. 3D-3F shows the structures of the closed prefusion conformation and two one-RBD-up conformation of the Kappa S trimer in ribbon diagram with the same protomers shown as in FIG. 3A. FIGS. 3G and 3H show the structures of the two one-RBD-up conformations of the Gamma S trimer in ribbon diagram with the same protomers shown as in FIG. 3A. All mutations in the three variants, as compared to the original virus (D614), are highlighted in the sphere model. FIG. 3I show structures, in the Delta closed trimer, of segments (residues 617-644) containing the 630 loop and segments (residues 823-862) containing the FPPR from each of the three protomers (a, b and c). The position of each RBD is indicated. Dashed lines indicate gaps in the chain trace (disordered loops).

FIGS. 4A-4D show the structural impact of mutations in the Delta S. FIG. 4A depicts superposition of the NTD structure of the Delta S trimer with the NTD of the G614 S trimer (PDB ID: 7KRQ). Locations of mutations T19R, G142D, E156G and deletion of F157 and R158 are indicated and these residues are shown in stick model. The N-terminal segment, 143-154 and 173-187 loops are rearranged between the two structures and highlighted in darker shading. FIG. 4B is the top view of the FIG. 4A. FIG. 4C shows the superposition of the RBD structure of the Delta S trimer with the RBD of the G614 S trimer. Locations of mutations L452R and T478K are indicated and these residues are shown in stick model. FIG. 4D is a close-up view of superposition of the Delta S2 with the S2 of the G614 S trimer near residue 950. Locations of the D950N mutation and charged residues in the vicinity including Lys947, Arg1014, Glu1017 from the protomer A and Glu773, Lys776, Glu780 and Arg1019 from the protomer B are indicated. All these residues are shown in stick model.

FIGS. 5A-5D show the superposition of the NTD structure of the Kappa S trimer with the NTD of the G614 S trimer. Locations of mutations E154K and Q218H, as well as Arg102 that forms a salt bridge with Glu154 in the G614 structure are indicated and these residues are shown in stick model. The 173-187 loop in the G614 trimer is highlighted in darker shading; it becomes disordered in the Kappa trimer. FIG. 5B shows the superposition of the RBD structure of the Kappa S trimer with the RBD of the G614 S trimer. Locations of mutations L452R and E484Q are indicated and these residues are shown in stick model. FIG. 5C depicts a view of superposition of the NTD structures of the Gamma and G614 (PDB ID: 7KRR) S trimers in the one-RBD-up conformation. Locations of mutations L18F, T20N, P26S, D138Y and R190S, as well as N-linked glycan attached to Asn20 in the Gamma structure are indicated and these residues are shown in stick model. FIG. 5D shows the superposition of the RBD structure of the Gamma S trimer with the RBD of the G614 S trimer. Locations of mutations K417T, E484K and N501Y are indicated and these residues are shown in stick model.

FIG. 6 depicts a schematic representation of full-length SARS-CoV-2 spike(S) proteins from the VOC Gamma (B.1.1.28), VOI Kappa (B.1.617.1) and VOC Delta (B.1.617.2). The sequences are derived from the Gamma (hCoV-19/Brazil/AM-992/2020), Kappa (hCoV-19/India/MH-NEERI-NGP-40449/2021) and Delta (hCoV-19/India/GJ-GBRC619/2021) variants. Segments of S1 and S2 include: NTD, N-terminal domain; RBD, receptor-binding domain; CTD1, C-terminal domain 1; CTD2, C-terminal domain 2; 630 loop; S1/S2, S1/S2 cleavage site; S2′, S2′ cleavage site; FP, fusion peptide; FPPR, fusion peptide proximal region; HR1, heptad repeat 1; CH, central helix region; CD, connector domain; HR2, heptad repeat 2; TM, transmembrane anchor; CT, cytoplasmic tail; and tree-like symbols for glycans. Positions of all mutations (from the amino-acid sequence of Wuhan-Hu-1) are shown.

FIGS. 7A-7B show the expression and cell-cell fusion of SARS-CoV-2 variant S proteins. FIG. 7A shows the expression and processing of the full-length S constructs in HEK293 cells. S samples prepared from HEK293 cells transiently transfected with 10 μg of the full-length S expression plasmids were detected by anti-RBD polyclonal antibodies. Bands for the uncleaved S and S1 fragment are indicated. FIG. 7B shows HEK293T cells transfected with the untagged, full-length S protein expression plasmids that were fused with ACE2-expressing cells. Cell-cell fusion led to reconstitution of α and ω fragments of β-galactosidase yielding an active enzyme, and the fusion activity was then quantified by a chemiluminescent assay. No ACE2 and no S were negative controls.

FIGS. 8A-8D show the comparison of membrane fusion mediated by the Delta variant with that by other variants. FIG. 8B shows the time-course of cell-cell fusion mediated by various full-length S proteins expressed at the 10 μg transfection level, as indicated, with HEK293 cells transfected with 10 μg of ACE2. FIG. 8B shows the time-course of cell-cell fusions mediated by various full-length S proteins (10 μg transfection), as indicated, with HEK293 cells with no exogenous ACE2. FIG. 8C shows the cell-cell fusions mediated by various full-length S proteins with HEK293 cells transfected with various levels (0-10 μg) of ACE2 expression constructs. FIG. 8D shows the time course of infection of HEK293-ACE2 cells by MLV-based pseudotyped viruses using various SARS-CoV-2 variant S constructs containing a CT deletion in a single cycle. Infection was initiated by mixing viruses and target cells, and viruses were washed out at each time point as indicated. Selected data points are highlighted in FIGS. 1A-1C. The experiments were repeated at least three times with independent samples giving similar results.

FIGS. 9A-9B show the production of full-length S protein from the Gamma, Kappa and Delta variants. FIG. 9A shows a strep-tag fused to the C-terminus of the full-length S protein by a flexible linker. FIG. 9B shows full-length S proteins extracted and purified in detergent DDM, and further resolved by gel-filtration chromatography on a Superose 6 column. Peak I, the prefusion S trimer; peak II, the postfusion S2 trimer; and peak III, the dissociated monomeric S1. Inset, peak fractions were analyzed by Coomassie stained SDS-PAGE. Labeled bands are S, S1and S2. Fr#, fraction number. Each experiment was repeated at least three times independently with similar results. The data for the preparations from the Wuhan-Hu-1 (D614) and B.1 (G614), published previously (28, 31), are included for convenient comparison.

FIG. 10 shows representative images of various full-length S trimers by negative stain EM.

FIGS. 11A-11C show additional antigenic properties of the purified full-length SARS-CoV-2 S proteins. FIG. 11A shows the antibody competition clusters as described in ref (32). Surface regions of the S trimer targeted by antibodies on S1 are highlighted by ellipses, including RBD-1, RBD-2, RBD-3, NTD-1 and NTD-2. The exact location of NTD-2 is uncertain and therefore marked with a dashed line. FIG. 11B shows the binding analysis of the prefusion S trimers from G614, Gamma, Kappa and Delta variants with soluble ACE2 constructs was performed by bio-layer interferometry (BLI). For ACE2 binding, the purified S proteins were immobilized on AR2G biosensors and dipped into the wells containing ACE2 at various concentrations. For antibody binding, various antibodies were immobilized to AHC biosensors and dipped into the wells containing each purified S protein at different concentrations. Binding kinetics were evaluated using a 1:1 Langmuir model except for antibody C12A2 targeting the RBD-2, which was analyzed by a bivalent binding model. The sensorgrams, as well as the fits, are shown. Binding constants highlighted by underlines were estimated by steady-state analysis as described in the Methods. RU, response unit. Binding constants are also summarized here and in Table 1. All experiments were repeated at least twice with essentially identical results. FIG. 11C shows the steady-state analysis by plotting steady-state responses against concentrations. Kd values were derived from the fits.

FIG. 12 shows the antigenic properties of the cell-surface S proteins assessed by flow cytometry. Antibody and ACE2 binding to the full-length S proteins of the G614, Gamma, Kappa and Delta variants, as well as an S2 construct expressed on the cell surfaces analyzed by flow cytometry. An S-delCT-Kappa with the C-terminal 19 residues deleted was included because the expression level of the full-length S construct (S-Kappa) was low. The antibodies and their targets are indicated. Two therapeutic anti-RBD antibodies by Regeneron, REGN10933 (casirivimab) and REGN10987 (imdevimab), were also included in this assay. A designed ACE2-based inhibitor ACE2615-foldon-T27W was used for detecting receptor binding (33). MFI, mean fluorescent intensity. The error bars represent standard errors of mean from measurements using three independently transfected cell samples. The flow cytometry assays were repeated three times with essentially identical results.

FIG. 13 shows the cryo-EM analysis of the Delta S trimer. Top, representative micrograph, and 2D averages (box dimension: 396 Å) of the cryo-EM particle images of the Delta S trimer. Bottom, data processing workflow for structure determination.

FIGS. 14A-14C shows the analysis of the Delta S trimer structure. FIG. 14A shows the 3D reconstructions of the Delta trimer preparation in the closed, two one-RBD-up conformations, respectively, and each are respresented according to local resolution estimated by ResMap. Angular distribution of the cryo-EM particles used in each reconstruction is shown in the side view of the EM map. FIG. 14B depicts the gold standard FSC curves of the three refined 3D reconstructions of the Delta S trimer. FIG. 14C is a representative density in gray surface from EM maps with a resolution better than 3.5 Å.

FIG. 15 shows the Cryo-EM analysis of the Kappa S trimer. Top, representative micrograph, and 2D averages (box dimension: 396 Å) of the cryo-EM particle images of the Kappa S trimer. Bottom, data processing workflow for structure determination.

FIGS. 16A-16C show the analysis of the Kappa S trimer structure. FIG. 16A shows 3D reconstructions of the Kappa S trimer preparation in the closed and two one-RBD-up conformations, respectively, and each are represented according to local resolution estimated by ResMap. Angular distribution of the cryo-EM particles used in reconstruction for the closed conformation is shown in the side view of the EM map. FIG. 16B depicts the gold standard FSC curves of the refined 3D reconstructions of the Kappa S trimer. FIG. 16C is a representative density in gray surface from EM maps with a resolution better than 3.5 Å.

FIG. 17 shows the Cryo-EM analysis of the Gamma S trimer. Top, representative micrograph, and 2D averages (box dimension: 396 Å) of the cryo-EM particle images of the Gamma S trimer. Bottom, data processing workflow for structure determination.

FIGS. 18A-18C show the analysis of the Gamma S trimer structure. FIG. 18A shows the 3D reconstructions of the Gamma S trimer preparation in the two one-RBD-up conformations, respectively, and each are represented according to local resolution estimated by ResMap. Angular distribution of the cryo-EM particles used in reconstruction for the closed conformation is shown in the side view of the EM map. FIG. 18B depicts the gold standard FSC curves of the refined 3D reconstructions of the Gamma S trimer. FIG. 18C is a representative density in gray surface from the 3.8 Å EM map.

FIG. 19 shows cryo-EM structures of the full-length S proteins of the Delta, Kappa and Gamma variants. Three structures of the Delta S trimer, representing a closed prefusion conformation and two distinct one-RBD-up conformations, were modeled based on corresponding cryo-EM density maps at 3.1-4.3 Å resolution. Three structures of the Kappa S trimer, representing a closed prefusion conformation and two distinct one-RBD-up conformations, were modeled based on corresponding cryo-EM density maps at 3.1-4.3A resolution. Two structures of the Gamma S trimer, representing two distinct one-RBD-up conformations, were modeled based on corresponding cryo-EM density maps at 3.8-4.4A resolution. The three protomers (a, b, c) are represetend. RBD locations are indicated. Particle percentage for each class in the data processing is also indicated, but it may not accurately reflect the conformation distribution of the S trimer in solution.

FIGS. 20A-20H show superposition of the variant trimer structures and the G614 structure. FIGS. 20A-20C show side views of superposition of the closed conformation and two distinct one-RBD-up conformations of the Delta S in ribbon diagram, aligned by the S2 portion with the closed prefusion structure and the one-RBD-up conformation of the G614 S, respectively. The positions of the RBD-down and three different RBD-up conformations are indicated. FIGS. 20D-20F show the comparison of the Kappa S and G614 trimer structures in the corresponding conformations, when aligned by S2. FIGS. 20G and 20H show the comparison of the Gamma S and G614 trimer structures in the one-RBD-up conformation, when aligned by S2.

FIG. 21 shows a comparison of the density of the N-linked glycan at Asn343 in the variant trimers. The EM maps of G614, Delta, Kappa, Alpha and Beta trimers in the closed prefusion conformation are compared at the same resolution (3.6 Å) and the same contour level.

FIG. 22 shows a modeled interface between the RBD and ACE2. The interface between ACE2 and RBD in ribbon diagram from the complex structure is shown (PDB ID: 6MOJ; ref (36). Modeled K417T, E484K, N501Y, E484Q, L452R and T478K are shown as sticks. Panels E484K and N501Y were published previously in ref (26).

FIGS. 23A-23D show the structural impact of the mutations in the variants. FIG. 23A shows superposition of the structure of the Kappa S trimer in ribbon representation with the structure of the G614 S, showing the regions near the mutation H1101D. FIG. 23B shows superposition of the NTD structures of the Gamma and G614 S trimers in the one-RBD-up conformation. Replacements of the N-terminal segment, 143-154 and 173-187 loops are indicated. FIGS. 23C and 23D show superposition of the structure of the Gamma S trimer in ribbon representation with the structure of the G614 S aligned by S2, showing the region near mutations H655Y and T1027I. All mutations are shown as sticks.

### DETAILED DESCRIPTION OF CERTAIN EMBODIMENTS

The terms “polypeptide” and “protein” are used interchangeably to refer to a polymer of amino acid residues linked by peptide bonds, and for the purposes of the instant disclosure, have a minimum length of at least 5 amino acids. Both full-length proteins and fragments thereof greater than 5 amino acids are encompassed by the definition. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage (e.g., cleavage by furins or metalloproteases), and the like. Furthermore, as used herein, a “polypeptide” or “protein” refers to a protein that includes modifications, such as deletions, additions, and substitutions (generally conservative in nature as would be known to a person in the art) to the native sequence, as long as the protein maintains the desired activity relevant to the purposes of the described methods. These modifications can be deliberate, as through site-directed mutagenesis, or can be accidental, such as through mutations of hosts that produce the proteins, or errors due to PCR amplification or other recombinant DNA methods.

As used herein, the term “oligomer,” when used in the context of a protein and/or polypeptide is intended to include, but is not limited to, a protein or polypeptide having at least two subunits. Oligomers include, but are not limited to, dimers, trimers, tetramers, pentamers, hexamers, heptamers, octamers, nonamers, decamers and the like.

As used herein, the term “oligomerization domain” refers to, but is not limited to, a polypeptide sequence that can be used to increase the stability of an oligomeric protein such as, e.g., to increase the stability of an ACE2 trimer or tetramer. Oligomerization domains may increase the stability of dimers, trimers, tetramers, pentamers, hexamers, heptamers, octamers, nonamers, decamers and larger oligomers. In certain aspects, oligomerization domains increase the stability of trimers. Oligomerization domains can be used to increase the stability of homooligomeric polypeptides as well as heterooligomeric polypeptides. Oligomerization domains are well known in the art. Examples of oligomerization domains include, but are not limited to, the T4-fibritin “foldon” trimer and streptavidin.

As used herein, the terms “bind,” “binding,” “interact,” and “interacting” refer to covalent interactions, noncovalent interactions and steric interactions. A covalent interaction is a chemical linkage between two atoms or radicals formed by the sharing of a pair of electrons (a single bond), two pairs of electrons (a double bond) or three pairs of electrons (a triple bond). Covalent interactions are also known in the art as electron pair interactions or electron pair bonds. Noncovalent interactions include, but are not limited to, van der Waals interactions, hydrogen bonds, weak chemical bonds (via short-range noncovalent forces), hydrophobic interactions, ionic bonds and the like. A review of noncovalent interactions can be found in Alberts et al., in Molecular Biology of the Cell, 3d edition, Garland Publishing, 1994. Steric interactions are generally understood to include those where the structure of the compound is such that it is capable of occupying a site by virtue of its three dimensional structure, as opposed to any attractive forces between the compound and the site.

The terms “subject,” and “patient,” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

In some embodiments, a sample is obtained from the subject or patient. Such samples include biological fluids or biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). Biological tissues may be processed to obtain cell suspension samples. The sample may also be a mixture of cells prepared in vitro. The sample may also be a cultured cell suspension. In case of the biological samples, the sample may be crude samples or processed samples that are obtained after various processing or preparation on the original samples. For example, various cell separation methods (e.g., magnetically activated cell sorting) may be applied to separate or enrich target cells from a body fluid sample such as blood.

Biological fluids or biological tissue can be collected using any of the standard methods known in the art. Obtaining a plasma sample from a subject means taking possession of a plasma sample of the subject. In some embodiments, the plasma sample may be removed from the subject by a medical practitioner (e.g., a doctor, nurse, or a clinical laboratory practitioner), and then provided to the person performing the measuring steps of the assay described herein. The plasma sample may be provided to the person performing the measuring steps by the subject or by a medical practitioner (e.g., a doctor, nurse, or a clinical laboratory practitioner). In some embodiments, the person performing the measuring steps obtains a plasma sample from the subject by removing a blood sample from the subject and isolating plasma from the blood sample.

A “diagnostically effective amount” of the compositions of the disclosure generally refers to an amount sufficient to detect the desired biological composition, e.g., detect the virion or a viral infection. Similarly, a “therapeutically effective amount” of the compositions of the disclosure generally refers to an amount sufficient to elicit the desired biological response, e.g., treat the condition. As will be appreciated by those of ordinary skill in this art, the effective amount as described herein may vary depending on such factors as the virus being detected, the method of detection, the condition being treated, the mode of administration, and the age, body composition, and health of the subject. A therapeutically effective amount need to be an amount required for clinical efficacy.

The terms “treat”, “treating”, “treatment”, and “therapy” encompass an action that occurs while a subject is suffering from a condition which reduces the severity of the condition (or a symptom associated with the condition) or retards or slows the progression of the condition (or a symptom associated with the condition).

In certain exemplary embodiments, vectors such as, for example, expression vectors, containing a nucleic acid encoding one or more ACE2 monomers described herein are provided. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably.

Techniques for determining nucleic acid and amino acid “sequence identity” are known in the art. Typically, such techniques include determining the nucleotide sequence of genomic DNA, mRNA or cDNA made from an mRNA for a gene and/or determining the amino acid sequence that it encodes, and comparing one or both of these sequences to a second nucleotide or amino acid sequence, as appropriate. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention.

### EXAMPLES

The invention now being generally described, it will be more readily understood by reference to the following examples, which are included merely for purposes of illustration of certain embodiments and embodiments of the present invention and are not intended to limit the invention.

### Example 1

The Delta variant of severe acute respiratory syndrome coronavirus 2 (SARS-CoV-2) has outcompeted previously prevalent variants and become a dominant strain worldwide. In some embodiments the structure, function and antigenicity of the Delta variant full-length spike(S) trimer is compared with those of other variants, including

Gamma, Kappa, and previously characterized Alpha and Beta. Delta S can fuse membranes more efficiently at low levels of cellular receptor ACE2 and its pseudotyped viruses infect target cells substantially faster than all other variants tested, possibly accounting for its heightened transmissibility. Mutations of each variant rearrange the antigenic surface of the N-terminal domain of the S protein in a unique way, but only cause local changes in the receptor-binding domain, consistent with greater resistance particular to neutralizing antibodies. These results advance the molecular understanding of distinct properties of these viruses and may guide intervention strategies.

### Membrane Fusion by Delta S is Substantially Faster Than That of Other Variants

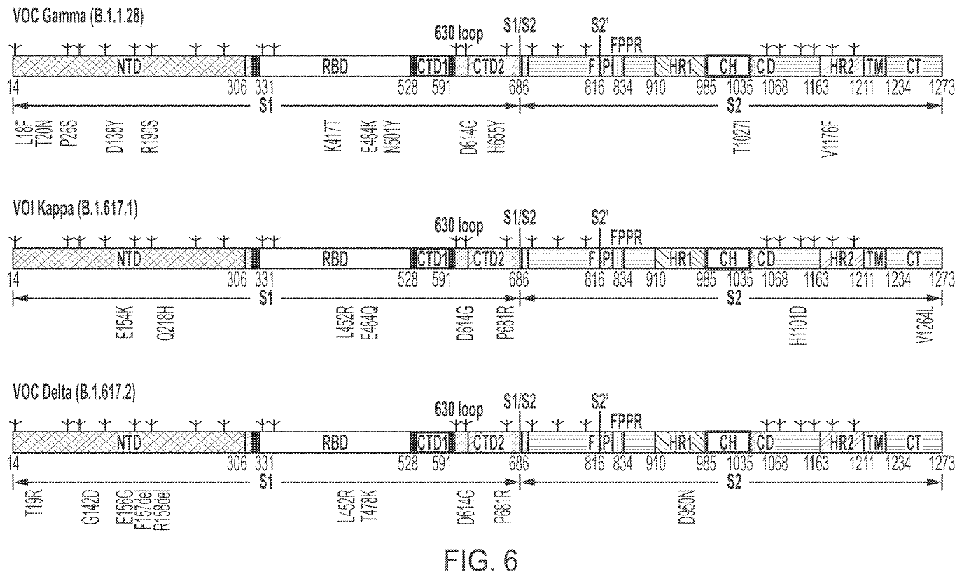

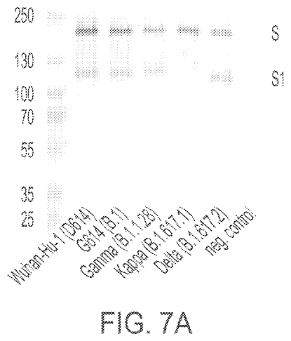

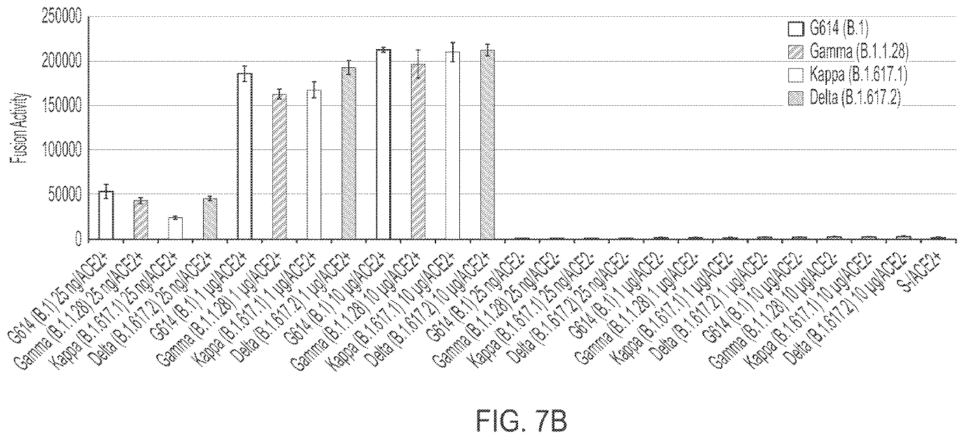

To characterize the full-length S proteins with the sequences derived from natural isolates of Gamma (hCoV-19/Brazil/AM-992/2020), Kappa (hCoV-19/India/MH-NEERI-NGP-40449/2021) and Delta (hCoV-19/India/GJ-GBRC619/2021) variants (FIG. 6), HEK293 cells were transfected with the respective expression constructs and their membrane fusion activities were compared with that of the full-length S construct of their parental strain (G614 or B.1 variant; ref (27)). All S proteins expressed at comparable levels (FIG. 7A). Kappa S showed a substantially lower level of cleavage between S1 and S2 than other variants, suggesting that a P681R mutation near the furin cleavage site does not increase the S protein processing. The same mutation, also present in the Delta variant, has not significantly altered the extent of cleavage from that seen in its parent strains (FIG. 7A). The cells producing these S proteins fused efficiently with ACE2-expressing cells (FIG. 7B). The fusion activity of Kappa S was ˜50% that of other S proteins at a low transfection level, but the difference diminished at high transfection levels (FIG. 7B).

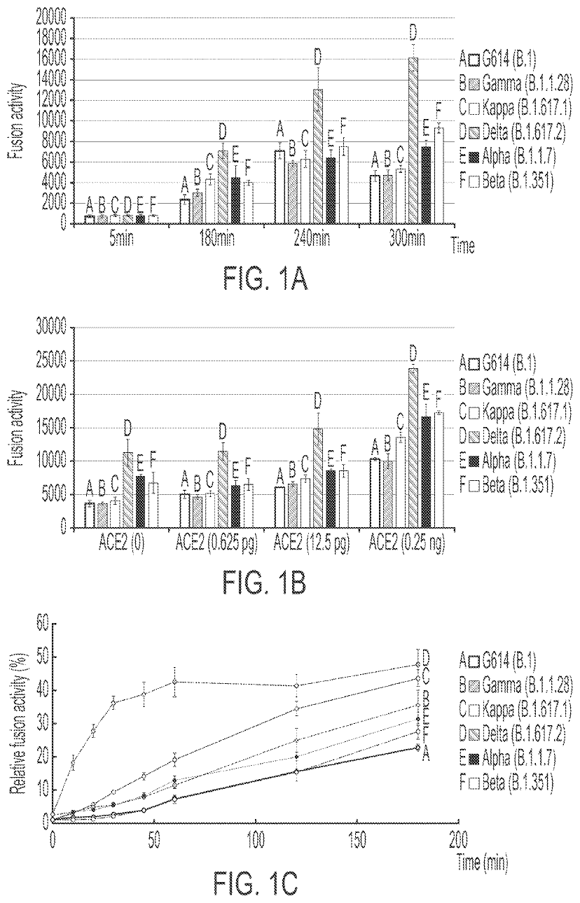

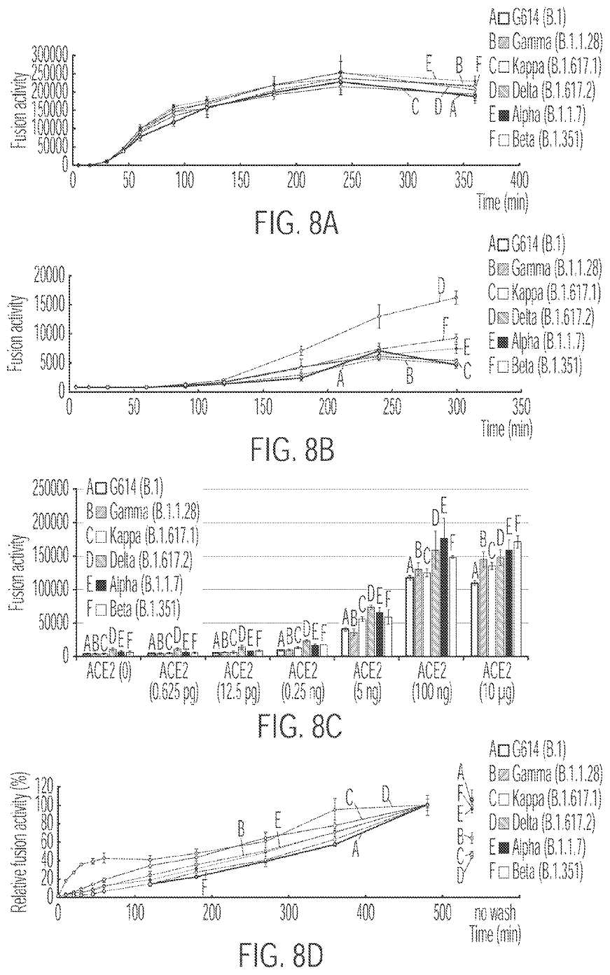

A time-course experiment with a cell-cell fusion assay was performed to determine if Delta S could fuse membranes more efficiently than other variants to account for its remarkable transmissibility. Both S and ACE2 transfected at high levels (FIG. 8A). No significant differences were found in the fusion activity among all the variants tested, including previously characterized Alpha and Beta (ref(26)). However, the Delta S-expressing cells fused with the negative-control HEK293 cells more efficiently than other variants, particularly at longer time points (FIGS. 1A and 8B). HEK293 cells express a minimal level of endogenous ACE2, and have been used as a negative control when not transfected by the ACE2 expression construct in prior 2-hour fusion protocols (ref(28)). The same pattern was also reproduced when small amounts of ACE2 were introduced in HEK293 cells, but the differences diminished when the ACE2 transfection level increased (FIGS. 1B and 8C). This data suggests Delta S could enter a host cell expressing low levels of ACE2 more efficiently than can other variants.

A similar time-course experiment was performed using murine leukemia virus (MLV)-based pseudoviruses expressing the S constructs with the cytoplasmic tail deleted to facilitate incorporation into particles (ref(29) and ref(30)). The infection was initiated by mixing the viruses and target cells and the viruses were washed out at each time point. The data in FIGS. 1C and 8D show that the Delta variant established infection much more rapidly in the first 60-minute period than did any other variant, when infectivity was normalized to its maximum level. The other variants gradually caught up over time, however, and eventually reached their maximum levels at 8 hours (FIG. 8D). Some viruses, including Delta, reproducibly showed lower measurements for the no wash-out controls than those measured at the 8-hour time point, consistent with some cytotoxicity that can reduce the expression of the reporter gene.

The data from both the cell-based and pseudovirus-based assays suggests that the Delta variant can infect a target cell substantially more rapidly than the other variants, either by more effective attachment or faster fusion kinetics.

### Biochemical and Antigenic Properties of the Intact S Proteins from the Variants

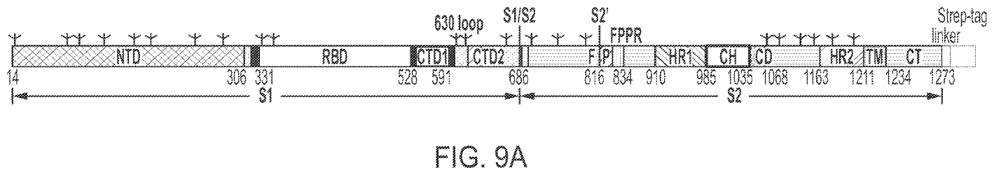

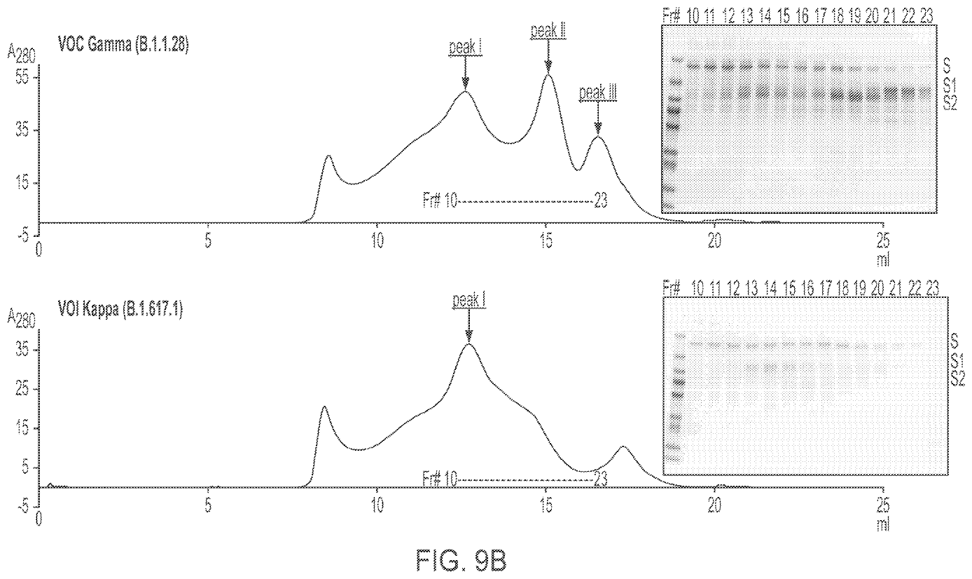

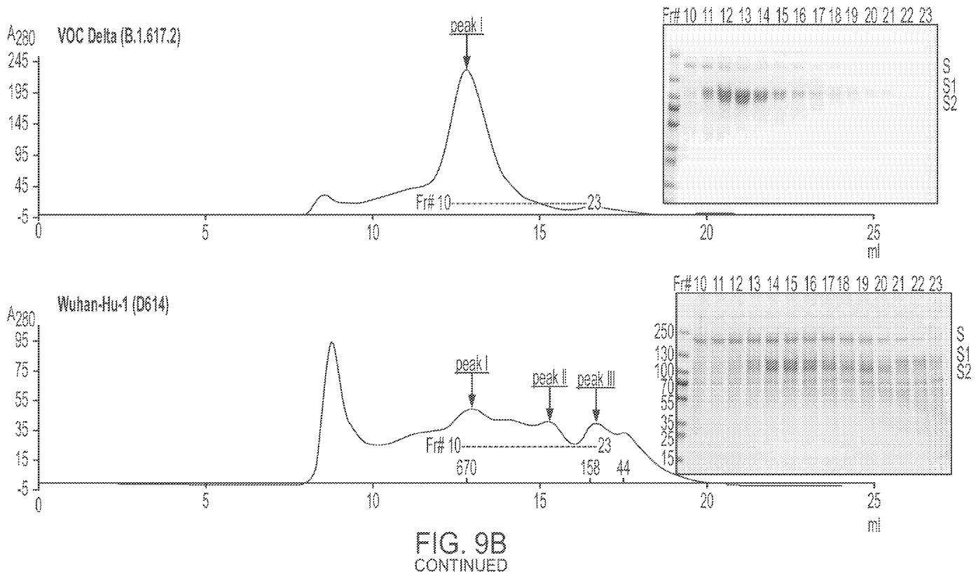

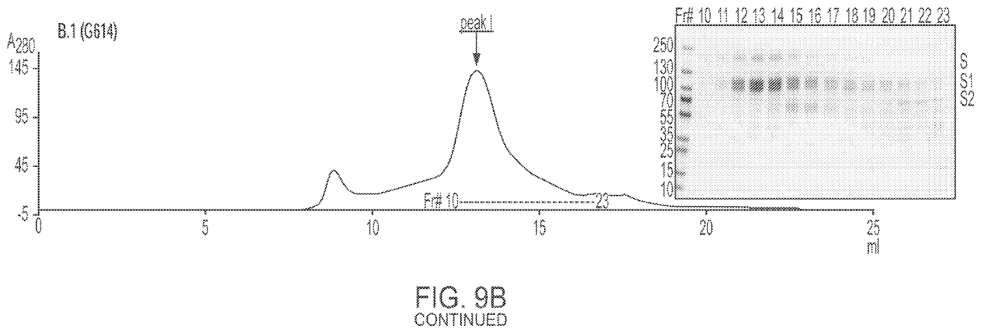

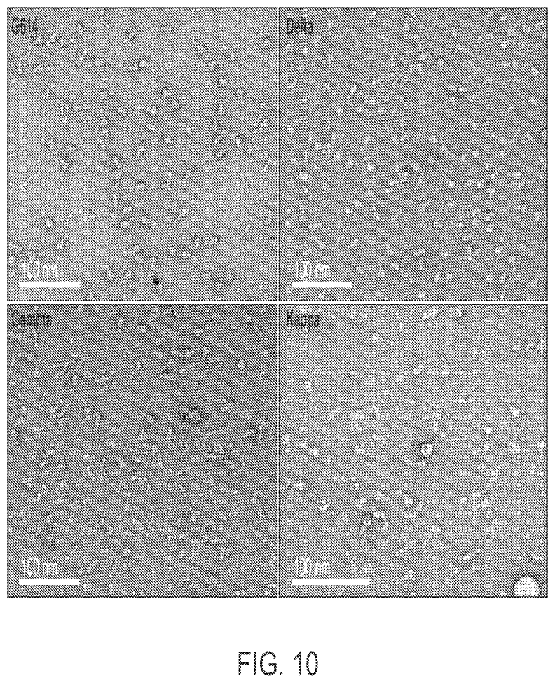

To analyze the full-length S proteins, a C-terminal strep-tag was added to the S proteins of the Gamma, Kappa and Delta variants (FIG. 9A), and the S proteins were expressed and purified by the procedures established for the D614, G614, Alpha and Beta S trimers (ref(26), ref(28), and ref(31)). The Gamma S protein eluted in three distinct peaks, corresponding to the prefusion S trimer, postfusion S2 trimer and dissociated S1 monomer, respectively (ref(28)), as shown by Coomassie-stained SDS-PAGE analysis (FIG. 9B). The prefusion trimer accounted for <40% of the total protein, like the profile of the original Wuhan-Hu-1 S protein, indicating that this trimer was not very stable. Although the Kappa protein eluted in one major peak corresponding to the prefusion trimer, there was a significant amount of aggregate on the leading side and a large shoulder on the trailing side, suggesting that the protein was also not very stable and conformationally heterogenous. Moreover, a large fraction of the protein remained uncleaved (FIG. 9B), further confirming that the furin cleavage was inefficient despite the P681R mutation. In contrast, the Delta S protein eluted in a single symmetrical peak of the prefusion trimer showing little aggregation or dissociation, and it was probably the most stable S trimer preparation among all the full-length S proteins that were examined (FIG. 8B; ref(31)). Negative stain EM also confirmed these results (FIG. 10). SDS-PAGE analysis showed that the Delta prefusion trimer peaks contained primarily the cleaved S1/S2 complex with a cleavage level very similar to that of the G614 and Beta S proteins (26, 31), again indicating little impact of the P681R mutation on the extent of furin cleavage.

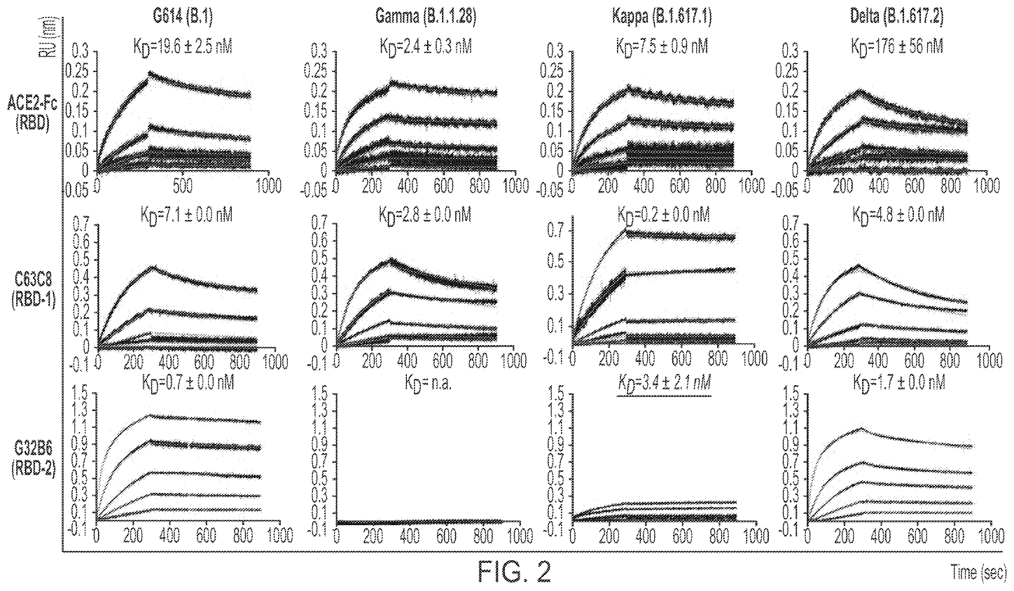

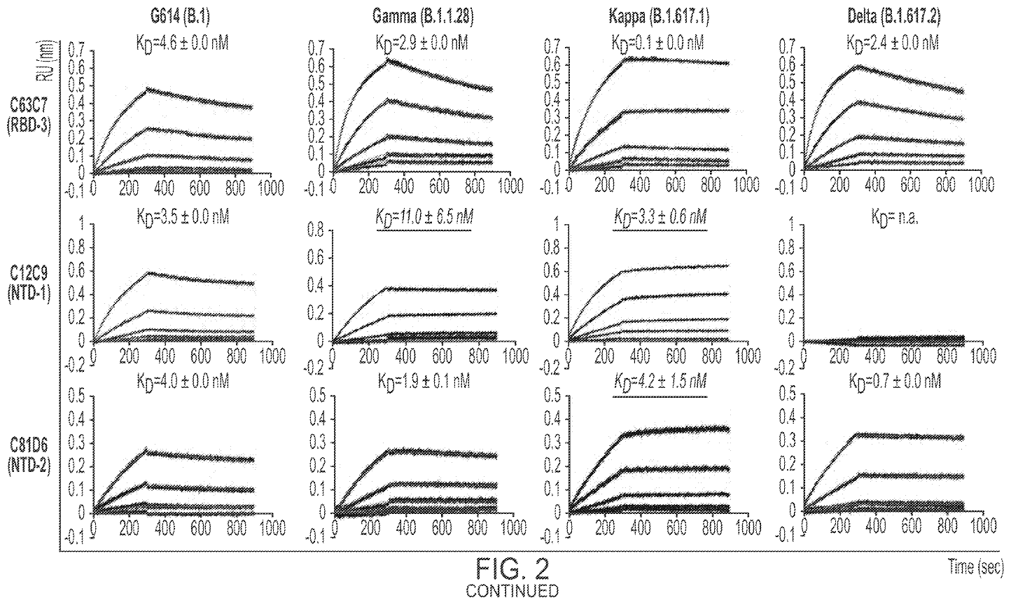

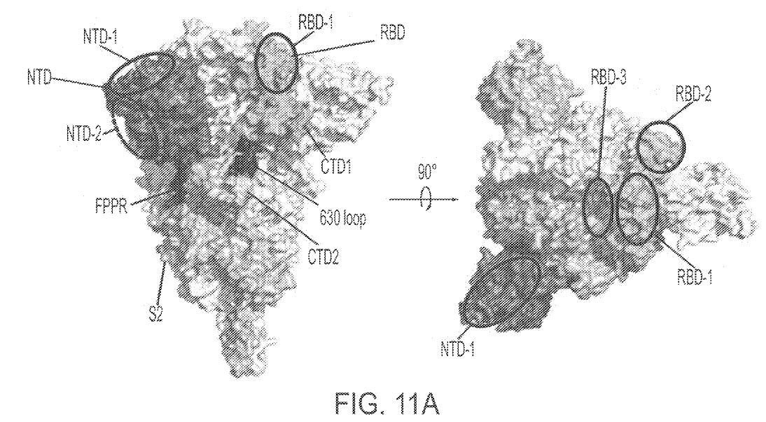

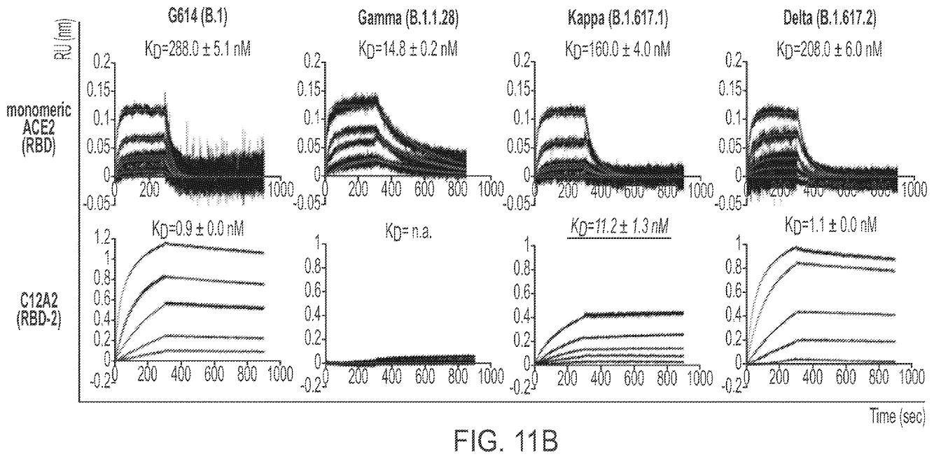

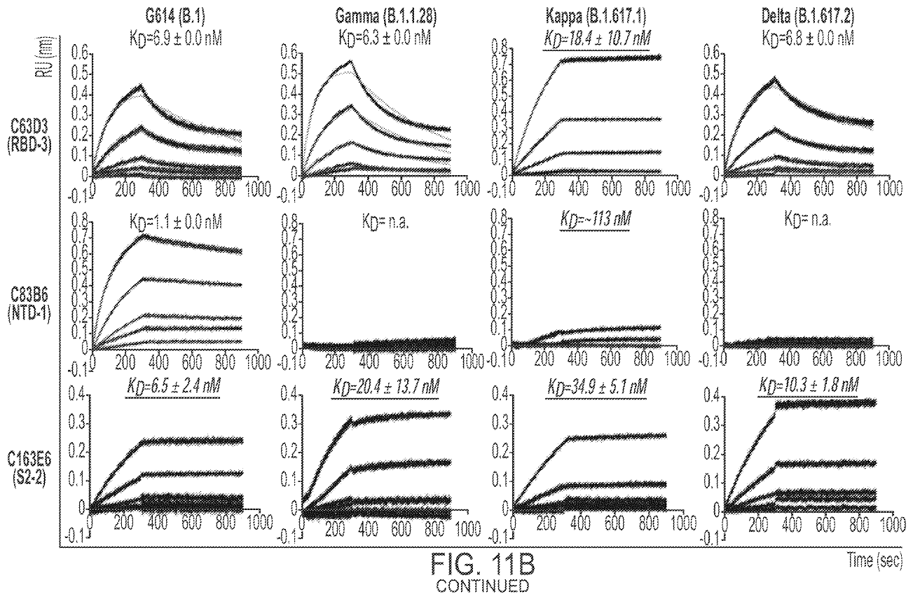

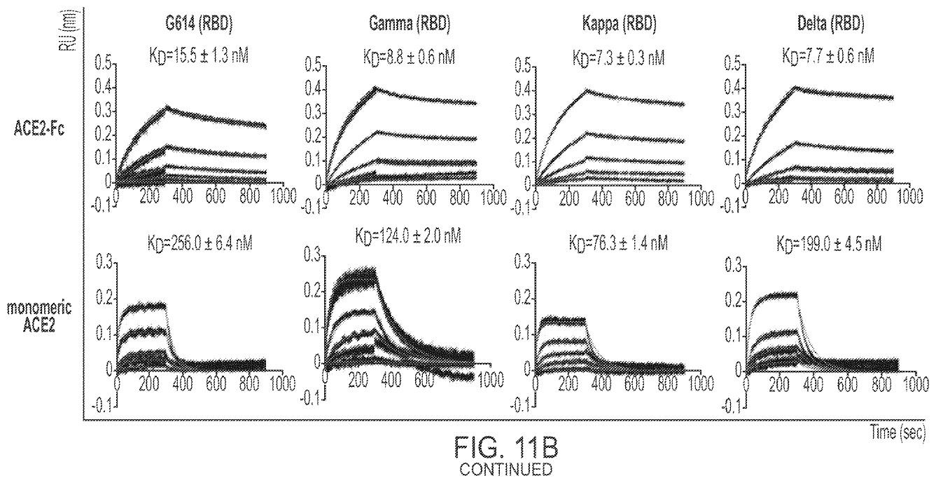

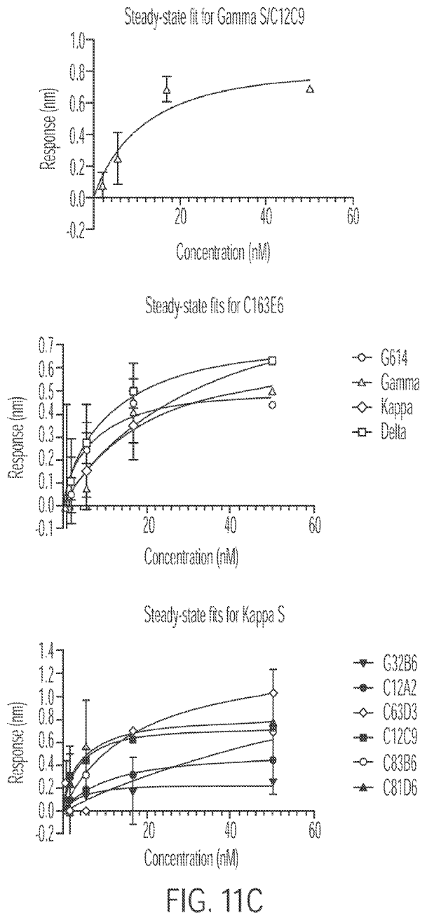

To analyze antigenic properties of the prefusion S trimers, the binding was measured to soluble ACE2 proteins and S-directed monoclonal antibodies isolated from COVID-19 convalescent individuals by bio-layer interferometry (BLI). The selected antibodies recognize distinct epitopic regions on the S trimer, as defined by competing groups designated RBD-1, RBD-2, RBD-3, NTD-1, NTD-2 and S2 (FIG. 11A; ref(32)). The last two groups contained primarily non-neutralizing antibodies. The Gamma variant bound more strongly to the receptor than did its G614 parent, regardless of the ACE2 oligomeric state (FIGS. 2 and 11B; Table 1), which could have been due to its mutations (K417T, E484K and N501Y) in the RBD. ACE2 affinities for the Kappa and Delta S proteins were intermediate between those of the G614 and Gamma trimers, with Kappa closer to the Gamma and Delta closer to G614, except for binding of Delta S trimer with dimeric ACE2, which had a higher off-rate than did the other variants (FIG. 2). These data were largely confirmed by using monomeric RBD preparations instead of the S trimers of these variants, except Kappa RBD showed slightly higher affinity for ACE2 than the Gamma RBD (FIG. 11B and Table 1). ACE2 did not dissociate more rapidly from the Delta RBD than it did from the Gamma and Kappa RBDs; a possible explanation for the apparently weaker affinity of the Delta trimer for the ACE2 dimer may be that ACE2 binding induces S1 dissociation. Overall, these results suggest that the mutations in the RBD of the Gamma variant enhance receptor recognition, while the RBD mutations in the Kappa (L452R and E484Q) and Delta (L452R and T478K) variants do not. The dimeric ACE2 appears to be more effective in inducing S1 dissociation from the Delta S trimer than from any other variant, including Alpha and Beta (26).

TABLE 1
Binding constants of S-ACE2 interaction

| | | K_{D} | K_{D} | ka | | ka | ka2 | kdis | | kdis | kdis2 |
|---|---|---|---|---|---|---|---|---|---|---|---|
| | | (M) | Error | (1/Ms) | ka2 | Error | Error | (1/s) | kdis2 | Error | Error |
| ACE2-Fc | G614 S | 1.96E−08 | 2.52E−09 | 2.64E+04 | 6.00E−01 | 2.29E+03 | 7.68E+00 | 5.18E−04 | 8.98E−02 | 4.91E−05 | 1.13E+00 |
| (RBD) | Gamma S | 2.41E−09 | 2.76E−10 | 9.45E+04 | 2.09E+01 | 4.95E+03 | 2.16E+00 | 2.28E−04 | 7.22E−01 | 2.31E−05 | 1.70E−01 |
| | Kappa S | 7.51E−09 | 8.99E−10 | 5.14E+04 | 4.29E−01 | 3.36E+03 | 5.44E−01 | 3.86E−04 | 3.85E−02 | 3.88E−05 | 5.59E−02 |
| | Delta S | 1.76E−07 | 5.64E−08 | 2.62E+04 | 1.06E−01 | 1.13E+03 | 4.38E−02 | 4.60E−03 | 1.83E−03 | 1.46E−03 | 4.41E−04 |
| | G614 | 1.55E−08 | 1.30E−09 | 2.83E+04 | 2.27E−03 | 2.14E+03 | 1.07E+03 | 4.40E−04 | 2.77E+00 | 1.60E−05 | 1.30E+06 |
| | RBD | | | | | | | | | | |
| | Gamma | 8.76E−09 | 5.67E−10 | 3.95E+04 | 4.08E+00 | 1.21E+03 | 8.31E+00 | 3.46E−04 | 5.48E−01 | 1.97E−05 | 1.11E+00 |
| | RBD | | | | | | | | | | |
| | Kappa | 7.34E−09 | 2.82E−10 | 4.02E+04 | 3.31E+00 | 1.10E+03 | 1.05E+02 | 2.95E−04 | 1.07E+00 | 7.95E−06 | 3.38E+01 |
| | RBD | | | | | | | | | | |
| | Delta | 7.66E−09 | 5.93E−10 | 2.64E+04 | 4.17E−02 | 1.72E+03 | 9.46E−01 | 2.02E−04 | 9.36E−02 | 8.40E−06 | 2.10E+00 |
| | RBD | | | | | | | | | | |
| Monomeric | G614 S | 2.88E−07 | 5.13E−09 | 1.00E+05 | | 1.71E+03 | | 2.89E−02 | | 1.48E−04 | |
| ACE2 | Gamma S | 1.48E−08 | 1.92E−10 | 2.20E+05 | | 2.75E+03 | | 3.26E−03 | | 1.10E−05 | |
| (RBD) | Kappa S | 1.60E−07 | 4.00E−09 | 9.71E+04 | | 2.31E+03 | | 1.55E−02 | | 1.22E−04 | |
| | Delta S | 2.08E−07 | 6.04E−09 | 7.38E+04 | | 2.05E+03 | | 1.53E−02 | | 1.33E−04 | |
| | G614 | 2.56E−07 | 6.44E−09 | 8.15E+04 | | 1.96E+03 | | 2.09E−02 | | 1.55E−04 | |
| | RBD | | | | | | | | | | |
| | Gamma | 1.24E−07 | 1.96E−09 | 5.68E+04 | | 8.58E+02 | | 7.06E−03 | | 3.16E−05 | |
| | RBD | | | | | | | | | | |
| | Kappa | 7.63E−08 | 1.38E−09 | 1.59E+05 | | 2.75E+03 | | 1.21E−02 | | 6.42E−05 | |
| | RBD | | | | | | | | | | |
| | Delta | 1.99E−07 | 4.46E−09 | 6.16E+04 | | 1.32E+03 | | 1.23E−02 | | 8.06E−05 | |
| | RBD | | | | | | | | | | |
| C63C8 | G614 S | 7.05E−09 | 4.38E−11 | 8.92E+04 | | 3.64E+02 | | 6.29E−04 | | 2.95E−06 | |
| (RBD-1) | Gamma S | 2.78E−09 | 1.56E−11 | 2.03E+05 | | 8.41E+02 | | 5.65E−04 | | 2.13E−06 | |
| | Kappa S | 1.90E−10 | 1.12E−11 | 1.31E+05 | | 5.07E+02 | | 2.50E−05 | | 1.47E−06 | |
| | Delta S | 4.76E−09 | 1.97E−11 | 1.95E+05 | | 6.97E+02 | | 9.29E−04 | | 1.94E−06 | |
| G32B6 | G614 S | 7.05E−10 | 2.13E−11 | 2.12E+05 | 2.76E+00 | 2.73E+03 | 1.09E−01 | 1.49E−04 | 3.22E−01 | 4.08E−06 | 2.31E−02 |
| (RBD-2) | Gamma S | N.D. | N.D. | N.D. | N.D. | N.D. | N.D. | N.D. | N.D. | N.D. | N.D. |
| | Kappa S | 3.43E−09^{a} | 2.09E−09 | | | | | | | | |
| | Delta S | 1.71E−09 | 2.10E−11 | 2.44E+05 | 3.22E+00 | 1.58E+03 | 5.29E+00 | 4.19E−04 | 6.43E−01 | 4.35E−06 | 1.05E+00 |
| C12A2 | G614 S | 9.00E−10 | 1.69E−11 | 2.01E+05 | 1.52E−01 | 1.94E+03 | 1.42E−02 | 1.81E−04 | 2.27E−02 | 2.93E−06 | 2.18E−03 |
| (RBD-2) | Gamma S | N.D. | N.D. | N.D. | N.D. | N.D. | N.D. | N.D. | N.D. | N.D. | N.D. |
| | Kappa S | 1.12E−08 | 1.33E−09 | | | | | | | | |
| | Delta S | 1.14E−09 | 1.65E−11 | 1.69E+05 | 1.10E−01 | 1.43E+03 | 1.39E−02 | 1.92E−04 | 2.73E−02 | 2.26E−06 | 3.61E−03 |
| C63C7 | G614 S | 4.64E−09 | 1.97E−11 | 9.09E+04 | | 3.04E+02 | | 4.22E−04 | | 1.10E−06 | |
| (RBD-3) | Gamma S | 2.86E−09 | 1.08E−11 | 1.65E+05 | | 4.44E+02 | | 4.72 E−04 | | 1.25E−06 | |
| | Kappa S | 1.18E−10 | 9.51E−12 | 1.04E+05 | | 3.06E+02 | | 1.23E−05 | | 9.87E−07 | |
| | Delta S | 2.36E−09 | 7.20E−12 | 1.94E+05 | | 4.01E+02 | | 4.58E−04 | | 1.03E−06 | |
| C63D3 | G614 S | 6.87E−09 | 4.45E−11 | 2.02E+05 | | 1.19E+03 | | 1.39E−03 | | 3.75E−06 | |
| (RBD-3) | Gamma S | 6.29E−09 | 3.40E−11 | 2.81E+05 | | 1.41E+03 | | 1.77E−03 | | 3.61E−06 | |
| | Kappa S | 1.84E−08 | 1.07E−08 | | | | | | | | |
| | Delta S | 6.82E−09 | 3.79E−11 | 1.63E+05 | | 8.10E+02 | | 1.11E−03 | | 2.80E−06 | |
| C12C9 | G614 S | 3.50E−09 | 1.91E−11 | 7.88E+04 | | 2.94E+02 | | 2.75E−04 | | 1.10E−06 | |
| (NTD-1) | Gamma S | 1.10E−08 | 6.48E−09 | | | | | | | | |
| | Kappa S | 3.31E−09 | 6.32E−10 | | | | | | | | |
| | Delta S | N.D. | N.D. | N.D. | | N.D. | | N.D | | N.D. | |
| C83B6 | G614 S | 1.07E−09 | 5.27E−12 | 1.86E+05 | | 3.60E+02 | | 1.99E−04 | | 9.03E−07 | |
| (NTD-1) | Gamma S | N.D. | N.D. | N.D. | | N.D. | | N.D. | | N.D. | |
| | Kappa S | ~1.13E−07 | N.A. | | | | | | | | |
| | Delta S | N.D. | N.D. | N.D. | | N.D. | | N.D. | | N.D. | |
| C81D6 | G614 S | 3.99E−09 | 4.74E−11 | 7.25E+04 | | 6.25E+02 | | 2.89E−04 | | 2.36E−06 | |
| (NTD-2) | Gamma S | 1.91E−09 | 5.92E−11 | 4.84E+04 | | 6.81E+02 | | 9.25E−05 | | 2.56E−06 | |
| | Kappa S | 4.16E−09 | 1.49E−09 | | | | | | | | |
| | Delta S | 6.87E−10 | 2.25E−11 | 7.02E+04 | | 4.30E+02 | | 4.83E−05 | | 1.56E−06 | |
| C163E6 | G614 S | 6.47E−09 | 2.38E−09 | | | | | | | | |
| (S2-2) | Gamma S | 2.04E−08 | 1.37E−08 | | | | | | | | |
| | Kappa S | 3.49E−08 | 5.11E−09 | | | | | | | | |
| | Delta S | 1.03E−08 | 1.75E−09 | | | | | | | | |

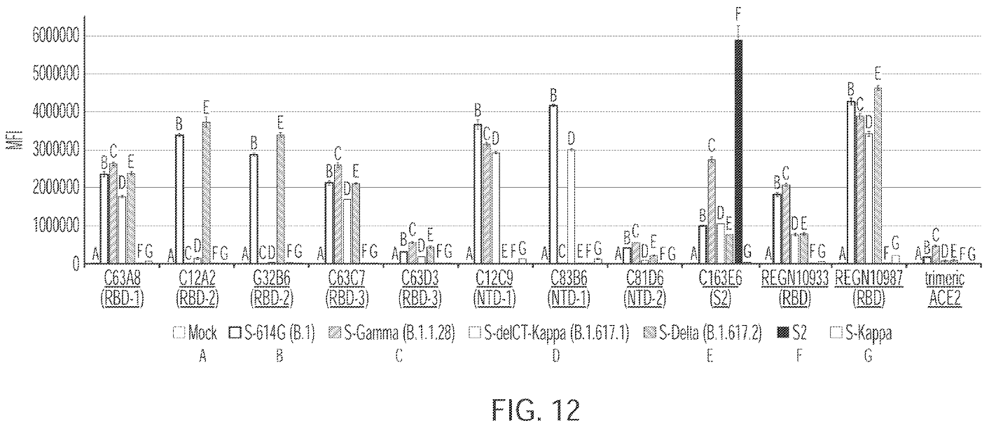

All selected monoclonal antibodies had reasonable affinities for the G614 S trimer (FIGS. 2 and 11B; Table 1). The Gamma variant completely lost binding to the two RBD-2 antibodies, G32B6 and C12A2, as well as to one NTD-1 antibody, C83B6, but it could still bind another NTD-1 antibody C12C9 with somewhat reduced affinity, suggesting that these two antibodies target two overlapping but distinct epitopes. Its affinities for the remaining antibodies were the same as those of the G614 trimer. Binding of the Kappa S trimer showed unrealistically slow off-rates for a number of antibodies (FIGS. 2 and 11B), presumably due to aggregation and conformational heterogeneity. Qualitatively, it had substantially weakened binding to the RBD-2 antibodies and the NTD-1 antibody C83B6, but with wildtype or even enhanced affinity for another NTD-1 antibody C12C9. Thus, the changes of its antigenic profile are similar to those of the Gamma S, but somewhat less complete. The Delta S only lost binding to the two NTD-1 antibodies with little changes in affinities for the other antibodies, including those targeting the RBD (FIGS. 2 and 11B; Table 1). The BLI data were also largely consistent with the binding results with the membrane-bound S trimers measured by flow cytometry (FIG. 12).

The neutralization potency of these antibodies and of trimeric ACE2 (33) was analyzed by measuring the extent to which they blocked infection by these variants in an HIV-based pseudovirus assay. For most antibodies, the neutralization potency correlated with their binding affinity for the membrane-bound or purified S proteins (Table 2). C81D6 and C163E6 recognize two non-neutralizing epitopes in the NTD and S2, respectively, and they did not neutralize any of the pseudoviruses. Thus, the mutations in the Gamma and Kappa variants have a greater impact on the antibody sensitivity of the virus than those in the Delta variant.

TABLE 2
Neutralization of the SARS-COV-2 variants

| Antibody/ | Neutralization titer (μg/ml) | | | | | | | |
|---|---|---|---|---|---|---|---|---|
| ACE2 | B.1 (G614) | | Gamma | | Kappa | | Delta | |
| construct | IC₅₀ | IC₅₀ | IC₅₀ | IC₅₀ | IC₅₀ | IC₅₀ | IC₅₀ | IC₅₀ |
| C63C8 | 8.030 | 27.794 | 4.378 | 24.127 | 5.619 | 25.555 | 4.563 | 19.939 |
| (RBD-1) | | | | | | | | |
| C12A2 | 0.014 | 0.071 | >50 | >50 | >50 | >50 | 0.063 | 0.339 |
| (RBD-2) | | | | | | | | |
| G32B6 | 0.018 | 0.051 | >50 | >50 | >50 | >50 | 0.015 | 0.048 |
| (RBD-2) | | | | | | | | |
| C63C7 | 4.729 | 29.985 | 3.273 | 18.781 | 4.532 | 20.257 | 7.704 | 34.343 |
| (RBD-3) | | | | | | | | |
| C63D3 | 37.186 | >50 | 30.614 | >50 | >50 | >50 | 46.126 | >50 |
| (RBD-3) | | | | | | | | |
| C12C9 | 0.019 | 43.34 | 0.341 | >50 | 22.939 | >50 | >50 | >50 |
| (NTD-1) | | | | | | | | |
| C83B6 | 0.105 | >50 | >50 | >50 | >50 | >50 | >50 | >50 |
| (NTD-1) | | | | | | | | |
| C81D6 | >50 | >50 | >50 | >50 | >50 | >50 | >50 | >50 |
| (NTD-2) | | | | | | | | |
| C163E6 | >50 | >50 | >50 | >50 | >50 | >50 | >50 | >50 |
| (S2-2) | | | | | | | | |
| ACE2- | 0.079 | 0.568 | 0.036 | 0.180 | 0.045 | 0.147 | 0.022 | 0.054 |
| T27W-Fd | | | | | | | | |
| Positive | 1,922 | 426 | 58 | 23 | 1,046 | 96 | 2,300 | 150 |
| serum pool_2 | | | | | | | | |
| (1/x) | | | | | | | | |
| Normal | <20 | <20 | <20 | <20 | <20 | <20 | <20 | <20 |
| Human | | | | | | | | |
| Serum (1/x) | | | | | | | | |

### Overall Structures of the Intact S Trimers of the Delta, Kappa and Gamma Variants

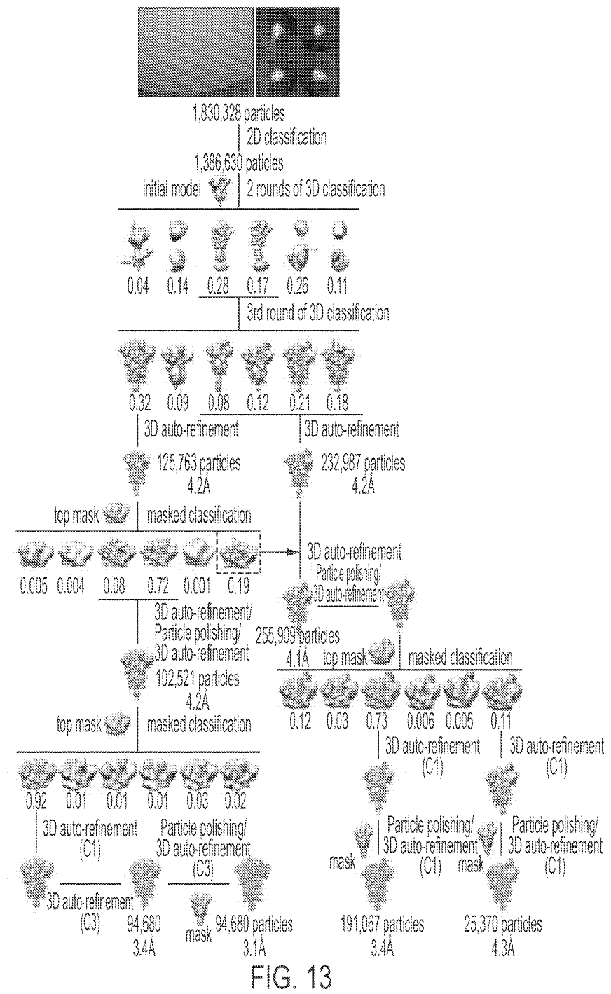

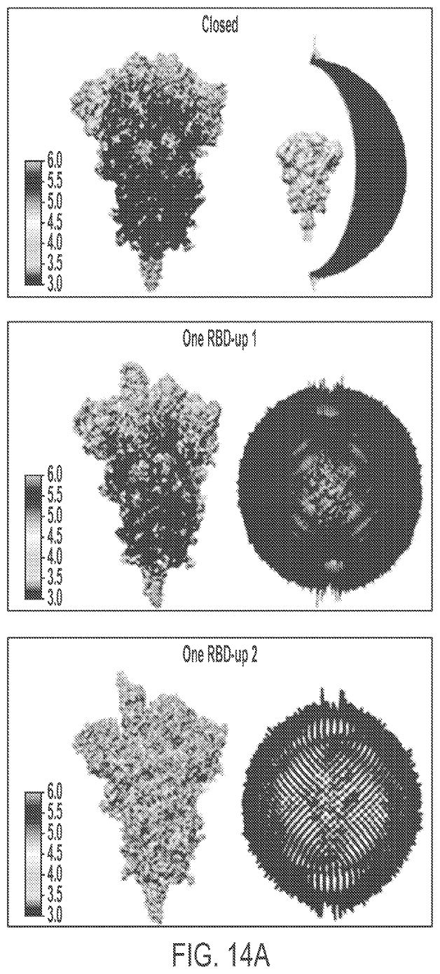

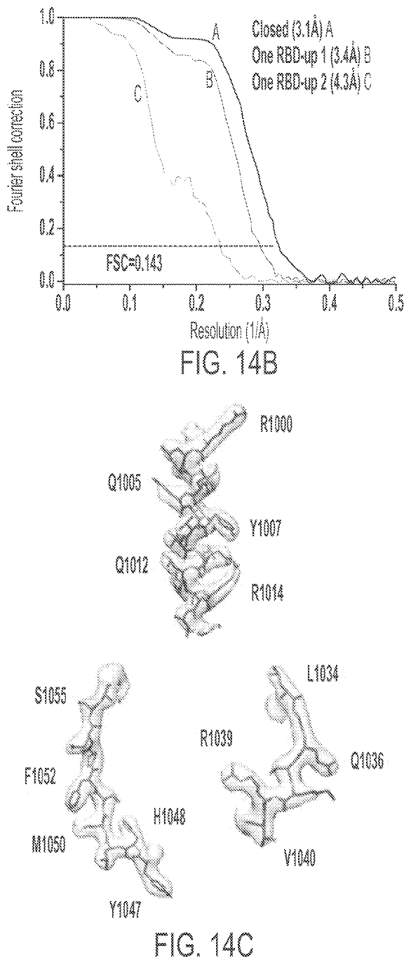

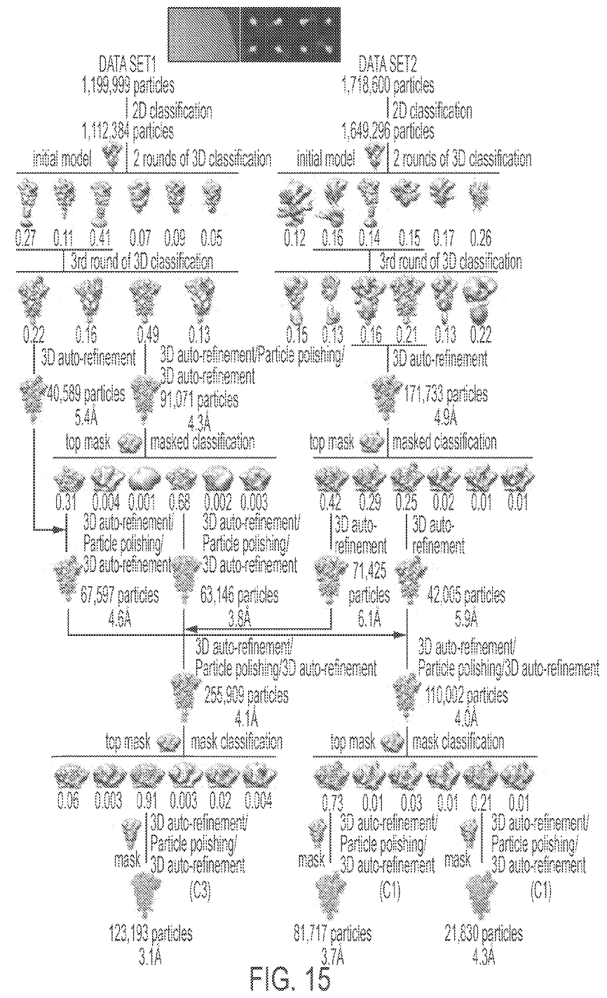

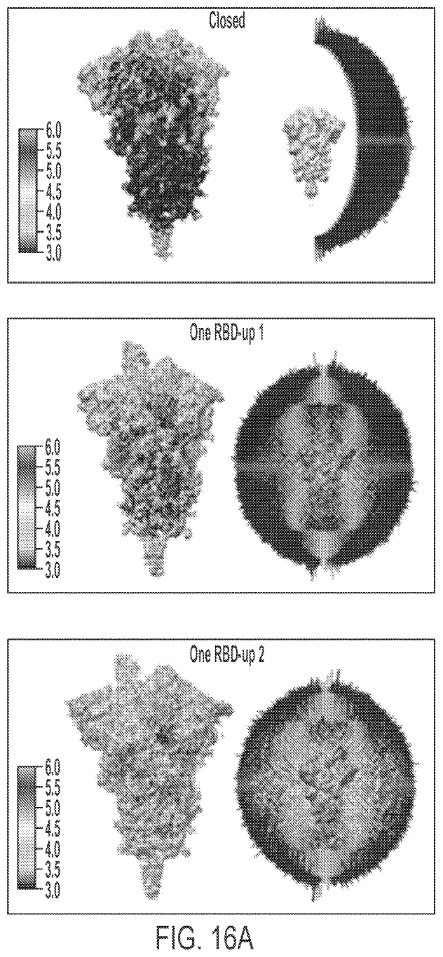

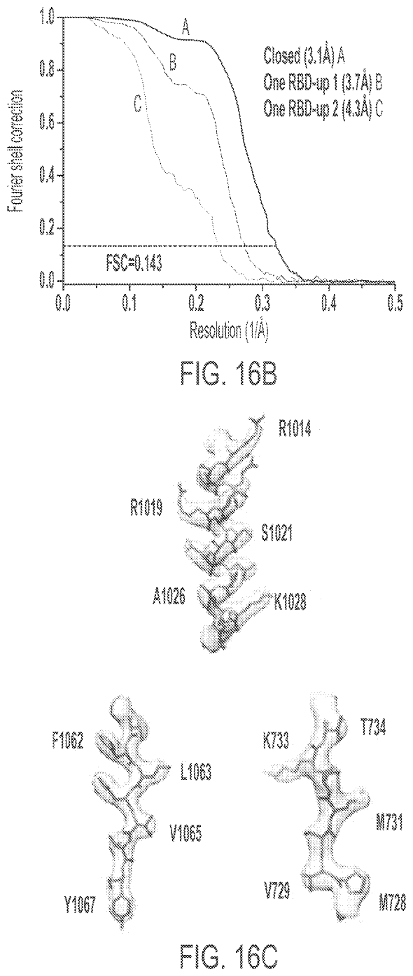

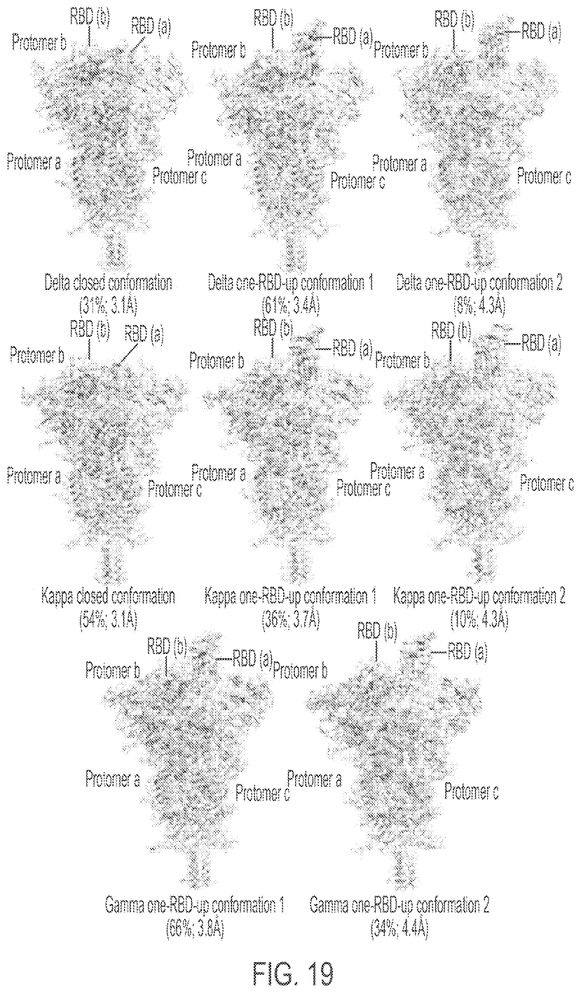

The cryo-EM structures of the full-length S trimers with the unmodified sequences of the Delta, Kappa and Gamma variants were identified. Cryo-EM images were acquired on a Titan Krios electron microscope equipped with a Gatan K3 direct electron detector. crYOLO (ref(34)) was used for particle picking, RELION (ref(35)) two-dimensional (2D) classification, three dimensional (3D) classification, and refinement (FIGS. 13-18C). 3D classification gave three distinct classes each for both the Delta and Kappa S trimers, representing one closed prefusion conformation and two one-RBD-up conformations, respectively. There were two different classes for the Gamma trimer, representing two one-RBD-up conformations. These structures were refined to 3.1-4.4 Å resolution (FIGS. 13-19; Table 3).

TABLE 3
Cryo-EM statistics.

| Protein | | |
|---|---|---|
| Full-length S of | Full-length S of | Full-length S of |
| Delta variant | Gamma variant | Kappa variant |

EM data collection and reconstruction statistics

| Microscope | Titan Krios | | | Titan Krios | | Titan Krios | | |
|---|---|---|---|---|---|---|---|---|
| Voltage(kV) | 300 | | | 300 | | 300 | | |
| Detector | Gatan K3 | | | Gatan K3 | | Gatan K3 | | |
| Magnification(nominal) | 105,000 | | | 105,000 | | 105,000 | | |
| Energy filter slit width (eV) | 20 | | | 20 | | 20 | | |
| Calibrated pixel size (Å/pix) | 0.825 | | | 0.825 | | 0.825 | | |
| Exposure rate (e⁻/pix/sec) | 20.24 | | | 20.69/20.63/27.13 | | 21.12/20,101 | | |
| Frames per exposure | 50 | | | 51/51/50 | | 51/51 | | |
| Total electron exposure (e⁻/Å²) | 51.48 | | | 54.72/54.56/53.4 | | 51.63/51.151 | | |
| Exposure per frame (e⁻/Å²) | 1.03 | | | 1.073/1.07/1.06 | | 1.012/1.003 | | |
| Defocus range (μm) | −0.8, −2.2 | | | −0.8, −2.3 | | −0.8, −2.2 | | |
| Automation software | SerialEM | | | SerialEM | | SerialEM | | |
| # of Micrographs used | 20,274 | | | 25,424/32,569/29,163 | | 22,019/17,314 | | |
| Particles extracted | 1,830,328 | | | 1,652,420/2,757,190/ | | 1,199,999/1,718,600 | | |
| | | | | 1,893,863 | | | | |
| Particles after 2D classification | 1,386,630 | | | 1,564,938/1,904,078/ | | 1,112,384/1,649,296 | | |
| | | | | 1,788,000 | | | | |
| Class | Closed | RBD-up 1 | RBD-up 2 | RBD-up 1 | RBD-up 2 | Closed | RBD-up 1 | RBD-up 2 |
| Total # of refined particles | 94,680 | 191,067 | 25,370 | 69,302 | 36,346 | 123,193 | 81,717 | 21,830 |
| Symmetry imposed | C3 | C1 | C1 | C1 | C1 | C3 | C1 | C1 |
| Estimated accuracy of | 0.87/1.96 | 1.23/2.45 | 2.35/4.24 | 1.73/3.11 | 2.27/3.80 | 1.04/2.29 | 1.66/3.24 | 1.86/3.36 |
| translations/rotations | | | | | | | | |
| Map sharpening B-factor | −93.6 | −111.9 | −92.1 | −124.6 | −153.7 | −103.6 | −121.8 | −130.3 |
| Unmasked Resolution at | 3.9/3.4 | 4.4/3.8 | 8.9/7.3 | 7.1/4.3 | 8.4/6.1 | 4.0/3.6 | 6.4/4.0 | 8.5/5.9 |
| 0.5/0.143 FSC (Å) | | | | | | | | |
| Masked resolution at 0.5/0.143 | 3.6/3.1 | 3.9/3.4 | 7.3/4.3 | 4.5/3.8 | 6.9/4.4 | 3.6/3.1 | 4.2/3.7 | 7.4/4.3 |
| FSC (Å) | | | | | | | | |

Model refinement and validation statistics

| | | | | | | | | |
|---|---|---|---|---|---|---|---|---|
| Class | Closed | RBD-up 1 | RBD-up 2 | RBD-up 1 | RBD-up 2 | Closed | RBD-up 1 | RBD-up 2 |
| PDB Composition | | | | | | | | |
| Amino acids | 3315 | 3287 | 3269 | 3230 | 3300 | 3330 | 3270 | 3270 |
| Glycans | 57 | 57 | 57 | 60 | 60 | 57 | 57 | 57 |
| RMSD bonds (Å) | 0.016 | 0.014 | 0.015 | 0.013 | 0.013 | 0.013 | 0.014 | 0.015 |
| RMSD angles (°) | 2.11 | 1.85 | 1.92 | 1.81 | 1.83 | 1.80 | 1.78 | 1.88 |
| Mean B-factors | | | | | | | | |
| Amino acids | 62 | 64 | 64 | 62 | 62 | 62 | 64 | 64 |
| Glycans | 92 | 88 | 88 | 88 | 88 | 92 | 88 | 87 |
| Ramachandran | | | | | | | | |
| Favored (%) | 92.33 | 92.63 | 91.68 | 92.16 | 91.96 | 93.09 | 93.07 | 91.58 |
| Allowed(%) | 6.48 | 6.64 | 7.42 | 7.17 | 7.16 | 6.06 | 6.40 | 7.77 |
| Outliers(%) | 1.19 | 0.74 | 0.90 | 0.67 | 0.89 | 0.85 | 0.53 | 0.65 |
| Rotamer outliers (%) | 5.92 | 4.40 | 3.86 | 2.07 | 1.84 | 2.41 | 1.89 | 2.38 |
| Clash score | 3.70 | 2.39 | 3.42 | 1.85 | 3.43 | 1.63 | 2.75 | 2.67 |
| C-beta outliers (%) | 1.23 | 0.81 | 1.01 | 048 | 0.49 | 0.35 | 0.33 | 0.39 |
| CaBLAM outliers (%) | 2.86 | 3.01 | 3.57 | 2.75 | 2.99 | 2.57 | 2.60 | 3.07 |
| CC (mask) | 0.78 | 0.76 | 0.61 | 0.69 | 0.63 | 0.79 | 0.73 | 0.63 |
| CC (volume) | 0.78 | 0.76 | 0.60 | 0.69 | 0.63 | 0.78 | 0.73 | 0.62 |
| MolProbity score | 2.22 | 1.97 | 2.08 | 1.67 | 1.82 | 1.65 | 1.72 | 1.84 |
| EMRinger score | 3.65 | 2.61 | 0.88 | 1.76 | 0.74 | 3.00 | 2.27 | 0.73 |

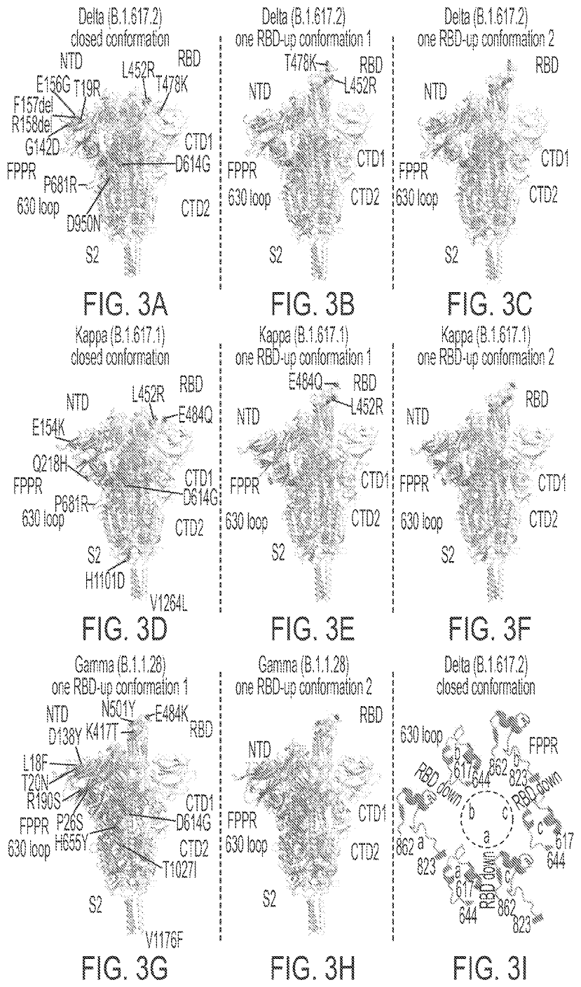

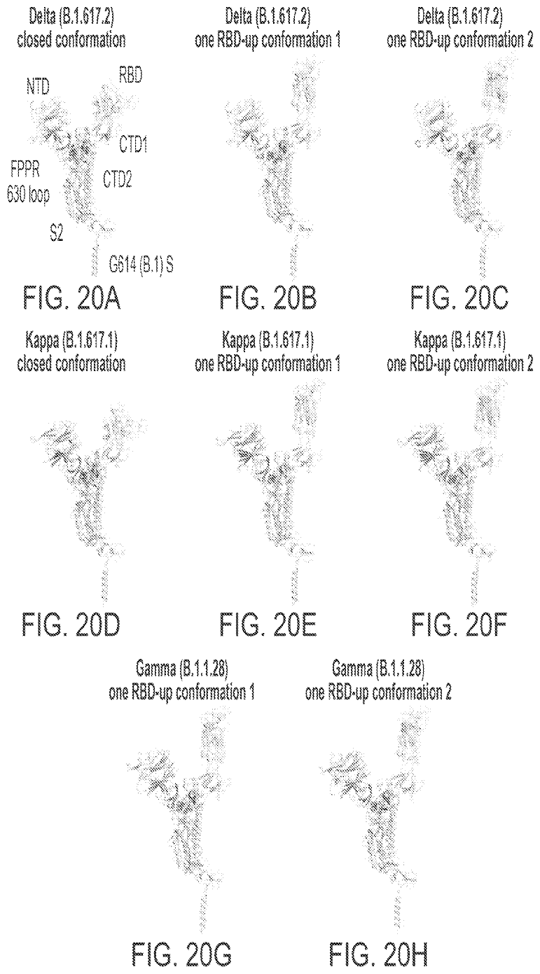

There were no major changes in the overall architectures of the full-length variant S proteins when compared to that of the parental G614 S trimer in the corresponding conformation (FIGS. 3 and 20A-20H; ref(31)). In the closed prefusion conformation, the NTD, RBD, CTD1 and CTD2 of S1 wrap around the S2 trimer. In the one-RBD-up conformation, the RBD movement did not cause any changes in the central helical core structure of S2, but opened up the trimer by shifting two adjacent NTDs away from the three-fold axis of the trimer. The furin cleavage site at the S1/S2 boundary (residues 682-685), including the P681R substitution, was still not visible in any these maps.

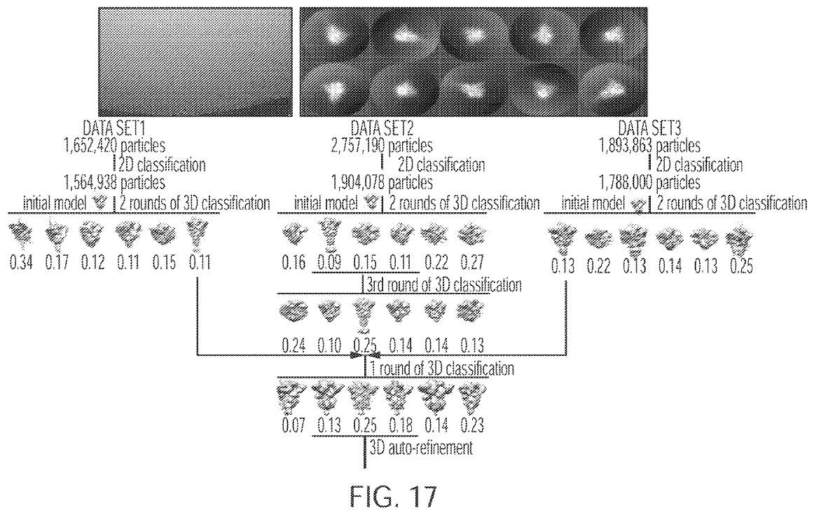

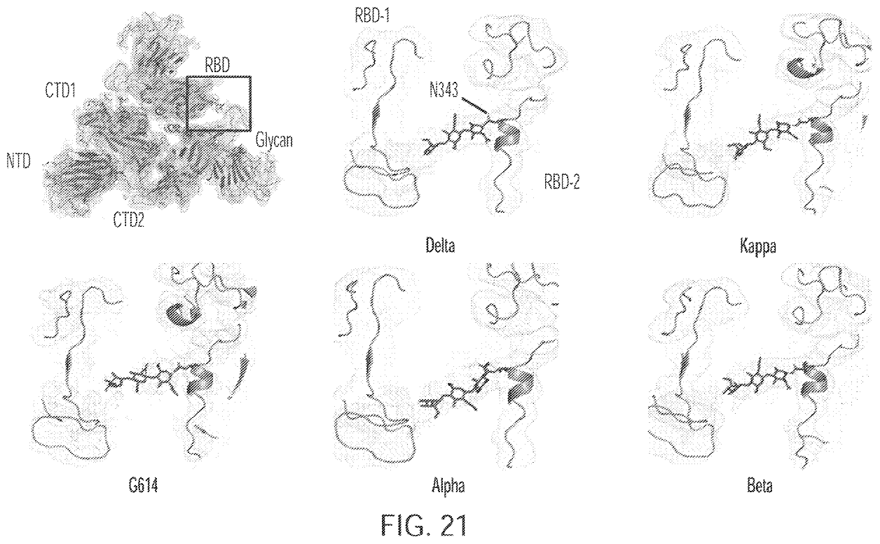

It is possible that the FPPR (fusion peptide proximal region; residues 828 to 853) and 630 loop (residues 620 to 640) are control elements and that shifts in their positions modulate the stability of the S protein and the kinetics of its structural rearrangements (28, 31). For the Delta and Kappa variants, the configurations of the FPPR and 630 loop are largely consistent with the distribution observed in the G614 trimer: all are structured in the RBD-down conformation, while only one the FPPR and 630-loop pair is ordered in the one-RBD-up conformations. The density of residues 841-847 in the FPPR of the Delta S in the closed prefusion state is weak probably because slight (1-2 Å) downward shifts of the CTD1 and RBD, which may weaken the packing of the FPPR (FIGS. 20A-20H). No class representing the closed conformation has been identified for the Gamma S from three independent data sets (FIG. 17), suggesting this conformational state was not well occupied by that variant, but one FPPR and 630-loop pair is structured in the one-RBD-up conformations of Gamma S, probably stabilizing the cleaved S trimers before receptor engagement. In all three variants, the distinct one-RBD-up structures differ only by the degree to which the up RBD and the adjacent NTD of its neighboring protomer shift away from the central threefold axis (FIG. 17). Density for an N-linked glycan at residue Asn343 in the RBD has become stronger in maps of all the new variants, particularly Delta and Kappa, than in that of the G614 trimer (FIG. 21). The distal end of the glycan appears to contact the neighboring RBD, forming a ring-like density to help clamp down the three RBDs. Nonetheless, it remains unclear why the Gamma prefusion trimer dissociates, the Kappa trimer tends to aggregate and the Delta trimer is the most stable of the three.

### Structural Consequences of Mutations in the Delta Variant

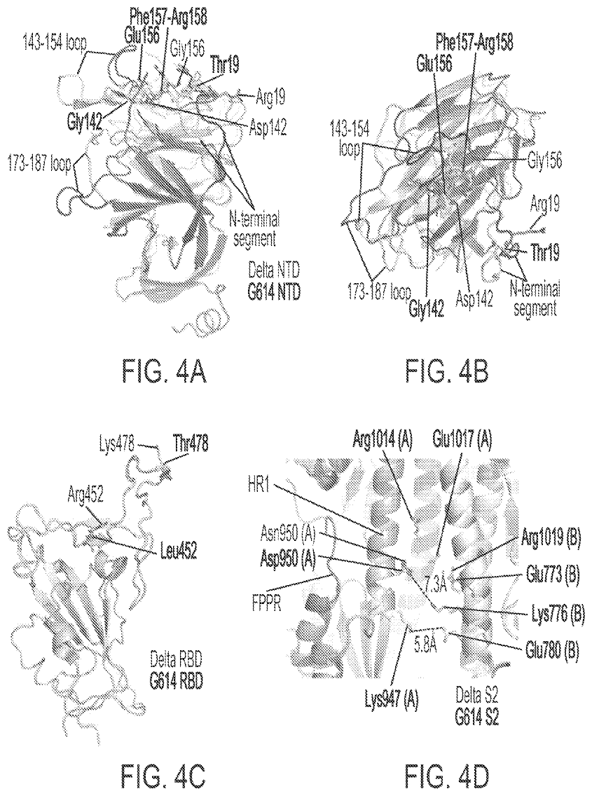

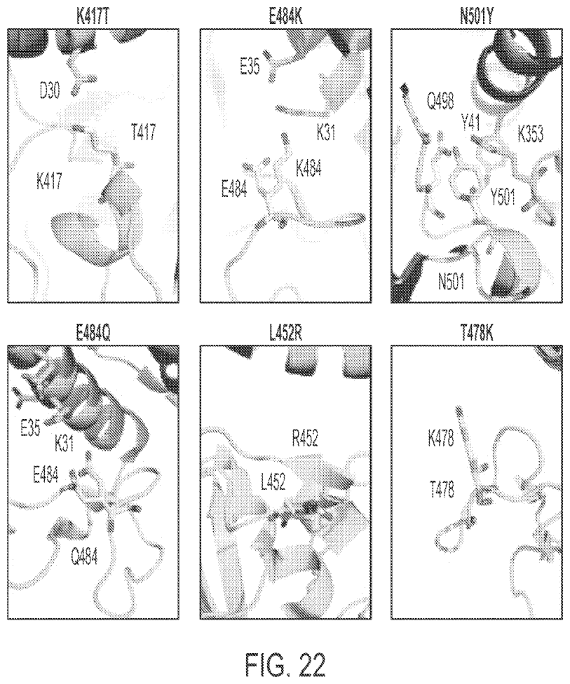

The structures of the Delta S trimer were superimposed onto the G614 trimer in the closed conformation aligning them by the S2 region (FIG. 17), revealing the most prominent differences in the NTD, which contains three point mutations (T19R, G142D and E156G) and a two-residue deletion (F157del and R158del). When the two NTDs were aligned (FIGS. 4A and 4B), it appeared that the mutations reshaped the 143-154 loop, projecting it away from the viral membrane together with an N-linked glycan (N149). They also reconfigured the N-terminal segment and the 173-187 loop, substantially altering the antigenic surface near the NTD-1 epitope group in the NTD. These structural changes are fully consistent with loss of binding and neutralization by NTD-1 antibodies (FIGS. 2 and 11A-11C; Table 2). There were two mutations, L452R and T478K, in the Delta RBD, which did not lead to any major structural rearrangements in the domain (FIG. 4C). These two residues were not in the ACE2 contacting surface, and it is therefore not surprising they had little impact on the receptor binding (FIG. 22). Neither binding nor neutralization of the Delta variant by most anti-RBD antibodies tested here changed, suggesting that the two residues are not in any major neutralizing epitopes either.

No obvious structural alterations were detected from the substitution D950N in S2 (FIG. 4D). This residue was in HR1 (heptad repeat 1) and also close to the FPPR. Although D950 in the G614 trimer is not close enough to form a salt bridge with any positively charged residue nearby, there were multiple pairs of charged residues in the vicinity that could help stabilize the packing between S2 protomers in the prefusion conformation. Thus, modifying the local electrostatic potential by the D950N substitution might introduce subtle changes that could influence the conformational changes of S2 required for membrane fusion.

### Structural Impact of the Mutations in the Kappa and Gamma Variants

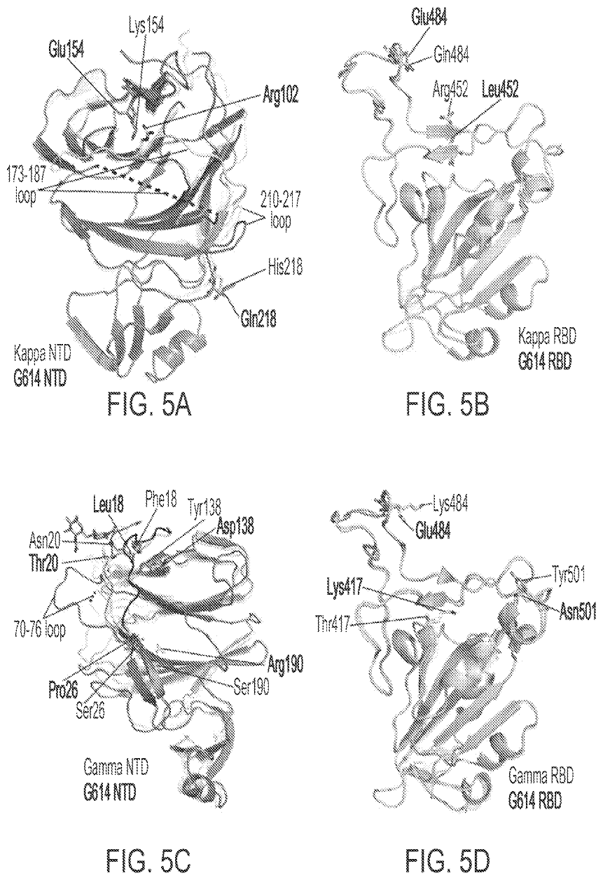

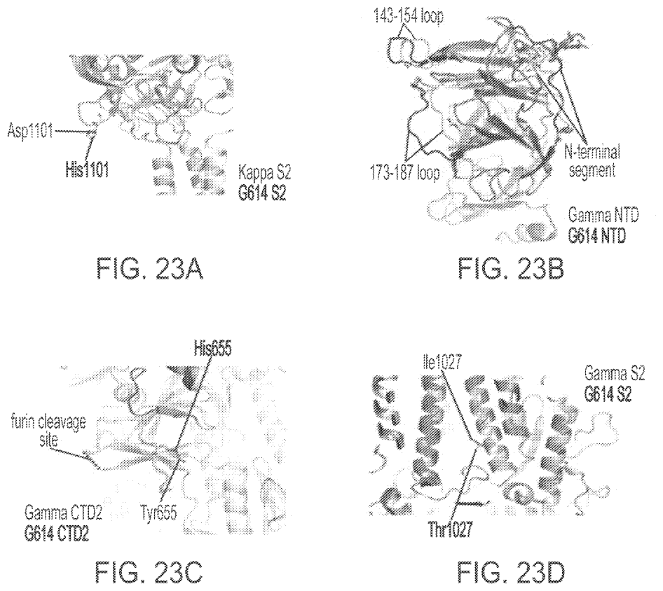

There were only two mutations (E154K and Q218H) in the NTD of the Kappa variant (FIG. 5A). In the G614 trimer, Glu154 formed a salt bridge with Arg102 (31). E154K substitution not only eliminateed the ionic interaction, but also exerted a repulsive force on Arg102, possibly impacting the nearby 173-187 loop, which was disordered in the Kappa trimer. Residue 218 was surface-exposed and on the opposite side from the neutralizing epitopes. Q218H appeared to shift the adjacent 210-217 loop and perhaps also contributed to the rearrangement of the 173-187 loop (FIG. 5A). Like the Delta RBD, there were two mutations (L452R and E484Q) in the Kappa RBD (FIG. 5B), which did not alter the overall structure of the domain. Glu484 could form a salt bridge with ACE2 Lys31 in the RBD-ACE2 complex (FIG. 22; ref(36) and ref(37)). The E484Q substitution loses the salt bridge, but hydrogen bonds between Glu484 and ACE2 Lys31 might compensate and thus account for a small increase in ACE2 binding affinity. L452R, also present in the Delta RBD, did not seem to have a significant impact on ACE2 binding (FIG. 22). In addition, the mutation H1101D in S2 caused little local changes (FIG. 23A), and V1264L was not visible in the structures.

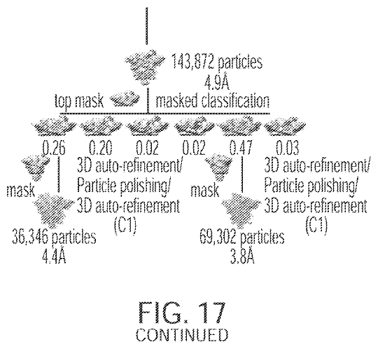

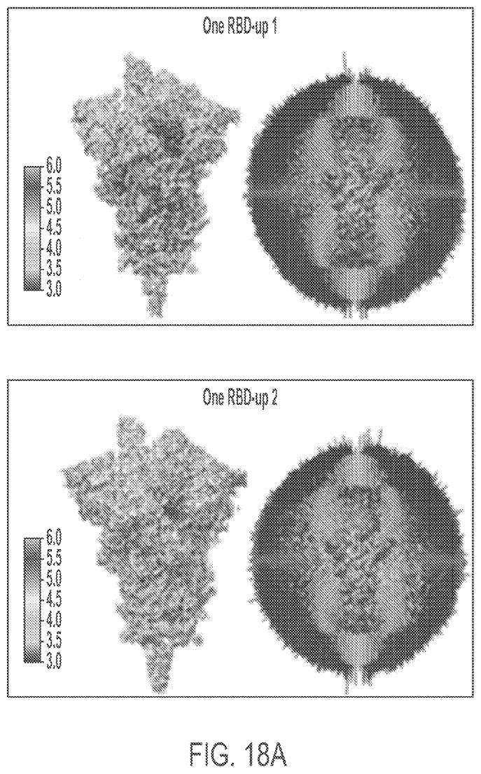

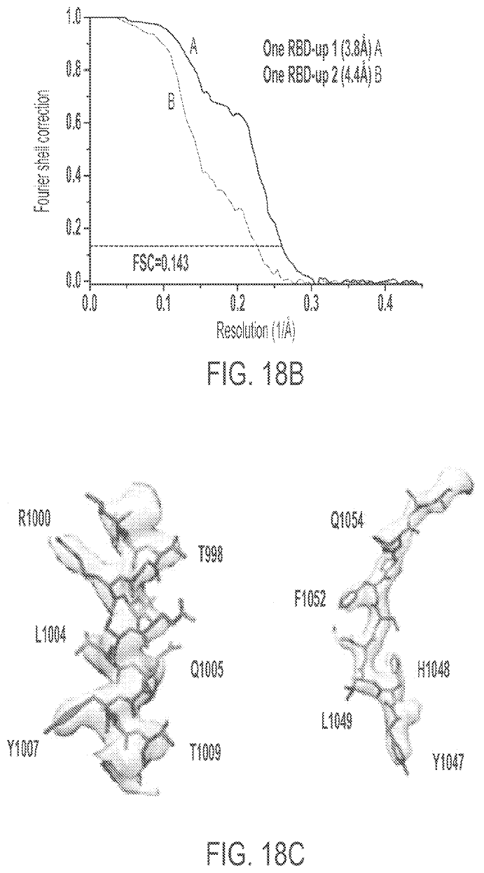

Three large independent data sets for the Gamma S protein were collected, but the number of good particles that could be extracted remained relatively low because of the instability and heterogeneity of the protein sample, giving two maps at 3.8 Å and 4.4 Å resolution, respectively (FIGS. 17 and 18A-18C). Nonetheless, the structural changes in the NTD caused by the mutations (L18F, T20N, P26S, D138Y and R190S) were evident even in these maps. All mutations except for R190S were near the N-terminal segment and contributed to reconfigure its extended structure (FIG. 5C). The new conformation of the N-terminal segment appeared to stabilize the 70-76 loop, disordered in most of SARS-CoV-2 S trimer structures published previously (ref(20), ref(28), and ref(38)). T2ON created a new glycosylation site and Asn20 was indeed glycosylated in the Gamma NTD protein (FIG. 5C). These changes apparently also shifted the 143-154 and 173-187 loops in the Gamma variant (FIG. 23B), leading to a relatively large-scale rearrangement of the antigenic surface of the NTD. Like the Beta RBD (ref(26), ref(39)), Gamma RBD had three mutations, K417T, E484K and N501Y, which did not produce any major structural rearrangements (FIG. 5D). N501Y increased receptor-binding affinity, which may be counteracted by K417T and E484K because of loss of ionic interactions with ACE2 (FIG. 22; ref(26) and ref(39)). K417T and E484K were probably responsible for loss of binding and neutralization of the Gamma by antibodies that target the RBD-2 epitopes. H655Y in the CTD2 did not change the local structure (FIG. 23C), but its location near the N-terminus of the cleaved S2 suggested that this mutation may play a role in destabilizing the S trimer of this variant. Finally, T1027I did not lead to any major changes in S2 (FIG. 23D), and V1176F was in a disordered region.

### Discussion

The Delta variant of SARS-CoV-2 has rapidly replaced the previously dominant variants, including Alpha, which is itself ˜60% more transmissible than the original Wuhan-Hu-1 strain (ref(40)-ref(42)). Delta thus appears to have acquired enhanced capacity for propagating in human cells. Several hypotheses have been proposed to explain its heighten transmissibility, including mutations in the RBD enhancing receptor engagement (ref(43)), P681R substitution near the S1/S2 boundary leading to more efficient furin cleavage (44, 45), and changes in its RNA polymerase increasing viral replication. It is possible that mutations in the viral replication machinery unique to the Delta variant (e.g. G671S in nsp12) may greatly increase the production of genomic RNA, but viral assembly into mature virions would require many other factors to achieve the >1,000 fold greater viral load in infected patients. No significant increase in ACE2 binding by either the full-length Delta S trimer or its RBD fragment was detected, nor was a more efficient cleavage observed in the Delta S than any other variants. Indeed, the P681R mutation was also present in the Kappa variant, which appears to have impaired furin cleavage, at least, in HEK293 cells used in the experiments. Two properties were identified, apparently unique to the Delta variant among those that have been studied so far, that might possibly account for its unusual transmissibility. First, when the Delta S protein was expressed on the cell surface at a saturating level, those cells fused more efficiently with target cells that produce low levels of ACE2 than did cells of any other variant, including previously characterized Alpha and Beta (ref(26)). When the ACE2 expression level increased, the differences among the variants diminished. Second, the pseudoviruses containing the Delta S construct entered the ACE2-expressing target cells substantially more rapidly than other variants. These data suggest that the Delta S protein has evolved to optimize the fusion step for entering cells expressing low levels of the receptor. This optimization may explain why the Delta variant can transmit upon relatively brief exposure and infect many more host cells rapidly, leading to a short incubation period and greater viral load during the infection.

The next consideration was what was the structural basis for the enhanced fusogenicity of the Delta S protein? All mutations but one in the Delta S were located in either the RBD or NTD. The extensive binding studies indicated that the Delta S did not engage the receptor ACE2 more tightly than any other variant. It was unclear what other functional roles the NTD may play in the membrane fusion process, besides protecting the nearby RBDs. If the mutations in the NTD enhanced RBD exposure to potential receptors, this should have been observed in the cryo-EM study as more particles in the RBD-up conformation from the Delta data set. Thus, the structural changes in both the RBD and NTD are unlikely to explain the efficient membrane fusion by the Delta variant. The last mutation, D950N, is unique to Delta and located in HR1 of S2 near the FPPR. D950N eliminates a negative charge (three in a trimer), but no obvious structural changes caused by this substitution in the prefusion conformation were observed. Its location nonetheless appears to be an important site that can influence the refolding of S2, required for membrane fusion. Although D950 is not involved in a salt bridge in the G614 trimer, it was conceivable that the local change in the electrostatic potential could destabilize the prefusion S2 in a very subtle way because there are several pairs of charged residues in the vicinity. Indeed, too much destabilization of the prefusion conformation may be detrimental since it could prompt the S trimer to undergo premature conformational changes and inactivate the protein. Thus, successful viral evolution must be a delicate balancing act to avoid tampering with its role in fusion.

The RBD and NTD are the two major sites on the S trimer targeted by neutralizing antibodies characterized previously (32, 46-48). The three strains described in some embodiments depict how different variants can use different strategies to remodel their NTD and evade host immunity. One important implication is that the function of the NTD does not require specific structural elements or sequences since the surface loops, β strands in the core structure and even some N-linked glycans can be rearranged in different ways without compromising viral infectivity. In contrast, the overall structure of the RBD has been strictly preserved among all the variants and reoccurring surface mutations appear to be limited to a number of sites (i.e. K417 in AY1 and AY2 sublineages of the Delta variant (rev(49)), consistent with its critical role in receptor binding. Therefore, it is possible that therapeutic antibodies or universal vaccines should not target the NTD since escaping from anti-NTD antibodies appears to be at little cost to the virus.

Continuous spread of SARS-CoV-2 worldwide will inevitably allow emergence of new variants as the virus evolves to survive under the selective pressure exerted by increasingly prevalent host immunity at the population level. Indeed, new sublineages of the Delta variant have been detected, including AY.1, AY.2 and AY.3 (rev(49)). The structure, function and antigenicity studies of the SARS-CoV-2 Delta, Kappa and Gamma variants show the molecular events that led these viruses to adapt in human communities and to evade host immunity. This analysis suggests a mechanism for the heightened transmissibility of the most contagious variant since the beginning of the SARS-CoV-2 outbreak.

### Materials and Methods

### Expression Constructs

Genes of full-length spike(S) protein from Gamma (hCoV-19/Brazil/AM-992/2020; GISAID accession ID: EPI_ISL_833172), Kappa (hCoV-19/India/MH-NEERI-NGP-40449/2021; GISAID accession ID: EPI_ISL_1547802) and Delta (hCoV-19/India/GJ-GBRC619/2021; GISAID accession ID: EPI_ISL_2020954) were synthesized by Twist Bioscience (South San Francisco, CA) or GENEWIZ (South Plainfield, NJ). The S genes were fused with a C-terminal twin Strep tag (SGGGSAWSHPQFEKGGGSGGGSGGSSAWSHPQFEK) (SEQ ID NO: 1) and cloned into a mammalian cell expression vector pCMV-IRES-puro (Codex BioSolutions, Inc, Gaithersburg, MD).

### Expression and Purification of Recombinant Proteins

Expression and purification of the full-length S proteins were carried out as previously described (28). Briefly, expi293F cells (ThermoFisher Scientific, Waltham, MA) were transiently transfected with the S protein expression constructs. To purify the S protein, the transfected cells were lysed in a solution containing Buffer A (100 mM Tris-HCl, pH 8.0, 150 mM NaCl, 1 mM EDTA) and 1% (w/v) n-dodecyl-B-D-maltopyranoside (DDM) (Anatrace, Inc. Maumee, OH), EDTA-free complete protease inhibitor cocktail (Roche, Basel, Switzerland), and incubated at 4° C. for one hour. After a clarifying spin, the supernatant was loaded on a strep-tactin column equilibrated with the lysis buffer. The column was then washed with 50 column volumes of Buffer A and 0.3% DDM, followed by additional washes with 50 column volumes of Buffer A and 0.1% DDM, and with 50 column volumes of Buffer A and 0.02% DDM. The S protein was eluted by Buffer A containing 0.02% DDM and 5 mM d-Desthiobiotin. The protein was further purified by gel filtration chromatography on a Superose 6 10/300 column (GE Healthcare, Chicago, IL) in a buffer containing 25 mM Tris-HCl, pH 7.5, 150 mM NaCl, 0.02% DDM. All RBD proteins were purchased from Sino Biological US Inc (Wayne, PA).

The monomeric ACE2 or dimeric ACE2 proteins were produced as described (33). Briefly, Expi293F cells transfected with monomeric ACE2 or dimeric ACE2 expression construct and the supernatant of the cell culture was collected. The monomeric ACE2 protein was purified by affinity chromatography using Ni Sepharose® excel (Cytiva Life Sciences, Marlborough, MA), followed by gel filtration chromatography. The dimeric ACE2 protein was purified by GammaBind™ Plus Sepharose beads (GE Healthcare), followed gel filtration chromatography on a Superdex 200 Increase 10/300 GL column. All the monoclonal antibodies were produced as described (32).

### Western Blot

Western blot was performed using an anti-SARS-COV-2 S antibody following a protocol described previously (50). Briefly, full-length S protein samples were prepared from cell pellets and resolved in 4-15% Mini-Protean® TGX™ gel (Bio-Rad, Hercules, CA) and transferred onto PVDF membranes. Membranes were blocked with 5% skimmed milk in PBS for 1 hour and incubated a SARS-CoV-2 (2019-nCOV) Spike RBD Antibody (Sino Biological Inc., Beijing, China, Cat: 40592-T62) for another hour at room temperature. Alkaline phosphatase conjugated anti-Rabbit IgG (1:5000) (Sigma-Aldrich, St. Louis, MO) was used as a secondary antibody. Proteins were visualized using one-step NBT/BCIP substrates (Promega, Madison, WI).

### Negative Stain EM

To prepare grids, 3 μl of freshly purified full-length S protein was adsorbed to a glow-discharged carbon-coated copper grid (Electron Microscopy Sciences), washed with deionized water, and stained with freshly prepared 1.5% uranyl formate. Images were recorded at room temperature at a magnification of 67,000× and a defocus value of 2.5 μm following low-dose procedures, using a Tecnai T12 electron microscope (Thermo Fisher Scientific) equipped with a Gatan UltraScan 895 4k CCD camera and operated at a voltage of 120 keV.

### Cell-Cell Fusion Assay

The cell-cell fusion assay, based on the α-complementation of E. coli β-galactosidase, was used to measure fusion activity of SARS-COV2 S proteins, as described (28). Briefly, HEK293T cells were transfected by polyethylenimine (PEI) (80 μg) with various amounts of the full-length SARS-COV2 (Wuhan-Hu-1, G614, Alpha, Beta, Gamma, Delta or Kappa) S construct, as indicated in each specific experiment (0.025-10 μg), and the a fragment of E. coli β-galactosidase construct (10 μg), as well as the empty vector to make up the total DNA amount to 20 μg, to generate S-expressing cells. The full-length ACE2 construct, as indicated in each specific experiment (0.625 pg-10 μg) together with the ω fragment of E. coli β-galactosidase construct (10 μg), and the empty vector when needed were used to transfect HEK293T cells to create target cells. After incubation at 37° C. for 24 hours, the cells were detached using PBS buffer and resuspended in complete DMEM medium. 50 μl S-expressing cells (1.0×106 cells/ml) were mixed with 50 μl ACE2-expressing target cells (1.0×106 cells/ml) to allow cell-cell fusion to proceed at 37° C. for from 5 minutes to 6 hours as indicated. Cell-cell fusion activity was quantified using a chemiluminescent assay system, Gal-Screen™ (Applied Biosystems, Foster City, CA), following the standard protocol recommended by the manufacturer. The substrate was added to the cell mixture and allowed to react for 90 minutes in dark at room temperature. The luminescence signal was recorded with a Synergy™ Neo plate reader (Biotek, Winooski, VT).

### Binding Assay by Bio-Layer Interferometry (BLI)

Binding of monomeric or dimeric ACE2 to the full-length Spike protein of each variant was measured using an Octet® RED384 system (ForteBio, Fremont, CA), following the protocol described previously (33). Briefly, a full-length S protein was immobilized to Amine Reactive 2nd Generation (AR2G) biosensors (ForteBio, Fremont, CA) and dipped in the wells containing the ACE2 protein at various concentrations (5.56-450 nM for monomeric ACE2; 0.926-75 nM for dimeric ACE2) for association for 5 minutes, followed by a 10-minute dissociation phase in a running buffer (PBS, 0.02% Tween 20, 2 mg/ml BSA). To measure binding of a full-length S protein to monoclonal antibodies, the antibody was immobilized to anti-human IgG Fc Capture (AHC) biosensor (ForteBio, Fremont, CA) following a protocol recommended by the manufacturer. The full-length S protein was diluted using a running buffer (PBS, 0.02% Tween 20, 0.02% DDM, 2 mg/ml BSA) to various concentrations (0.617-50 nM) and transferred to a 96-well plate. The sensors were dipped in the wells containing the S protein solutions for 5 minutes, followed with a 10-minute dissociation phase in the running buffer. Control sensors with no S protein or antibody were also dipped in the ACE2 or S protein solutions and the running buffer as references. Recorded sensorgrams with background subtracted from the references were analyzed using the software Octet® Data Analysis HT Version 12.0 (ForteBio). Binding kinetics was evaluated using a 1:1 Langmuir model except for dimeric ACE2 and antibodies G32B6 and C12A2, which were analyzed by a bivalent binding model. Sensorgrams showing unrealistic off-rates were fit individually to a single exponential function shown below to obtain the steady state response Req at each concentration.

R=Req*(1−e\hat{ }(−k*t))

The Kd was obtained by fitting Req value and its corresponding concentration to the model: “one site-specific” using GraphPad Prism 8.0.2 according to H. J. Motulsky, Prism 5 Statistics Guide, 2007, GraphPad Software Inc., San Diego CA, www.graphpad.com).

### Flow Cytometry

Expi293F cells (ThermoFisher Scientific) were grown in Expi293 expression medium (ThermoFisher Scientific). Cell surface display DNA constructs for the SARS-CoV-2 spike variants together with a plasmid expressing blue fluorescent protein (BFP) were transiently transfected into Expi293F cells using ExpiFectamine 293 reagent (ThermoFisher Scientific) per manufacturer's instruction. Two days after transfection, the cells were stained with primary antibodies or the histagged ACE2615-foldon T27W protein (33) at 10 μg/ml concentration. For antibody staining, an Alexa Fluor 647 conjugated donkey anti-human IgG Fc F(ab′)2 fragment (Jackson ImmunoResearch, West Grove, PA) was used as secondary antibody at 5 μg/ml concentration. For ACE2615-foldon T27W staining, APC conjugated anti-HIS antibody (Miltenyi Biotec, Auburn, CA) was used as secondary antibody at 1:50 dilution. Cells were run through an Intellicyt iQue® Screener Plus flow cytometer. Cells gated for positive BFP expression were analyzed for antibody and ACE2615-foldon T27W binding. The flow cytometry assays were repeated three times with essentially identical results.

### MLV-Based Pseudovirus Assay

Murine Leukemia Virus (MLV) particles, pseudotyped with various SARS-CoV-2 S protein constructs, were generated in HEK 293T cells, following a protocol described previously for SARS-CoV (51, 52). To enhance incorporation of S protein into the particles, the C-terminal 19 residues in the cytoplasmic tail of each S protein were deleted. To prepare for infection, 7.5×10³ of HEK 293 cells, stably transfected with a full-length human ACE2 expression construct, in 15 μl culture medium were plated into a 384-well white-clear plate coated with poly-D-Lysine to enhance the cell attachment. On day 2, 15 μl of MLV pseudoviruses for each variant were added into each well pre-seeded with HEK293-ACE2 cells. The plate was centrifuged at 114×g for 5 minutes at 12° C. After incubation of the pseudoviruses with the cells for a time period (10 minutes-8 hours), as indicated in the figures, the medium was removed and the cells were washed once with 1×DPBS. 30 μl of fresh medium was added back into each well. The cells were then incubated at 37° C. for additional 40 hours. Luciferase activities were measured with Firefly Luciferase Assay Kit (CB-80552-010, Codex BioSolutions Inc).

### HIV-Based Pseudovirus Assay

Neutralizing activity against SARS-CoV-2 pseudovirus was measured using a single-round infection assay in 293T/ACE2 target cells. Pseudotyped virus particles were produced in 293T/17 cells (ATCC) by co-transfection of plasmids encoding codon-optimized SARS-CoV-2 full-length S constructs, packaging plasmid pCMV DR8.2, and luciferase reporter plasmid pHR'CMV-Luc. For neutralization assays, serial dilutions of monoclonal antibodies (mAbs) were performed in duplicate followed by addition of pseudovirus. Pooled serum samples from convalescent COVID-19 patients or pre-pandemic normal healthy serum (NHS) were used as positive and negative controls, respectively. Plates were incubated for 1 hour at 37° C. followed by addition of 293/ACE2 target cells (1×10⁴/well). Wells containing cells+pseudovirus (without sample) or cells alone acted as positive and negative infection controls, respectively. Assays were harvested on day 3 using Promega BrightGlo™ luciferase reagent and luminescence detected with a Promega GloMax® luminometer. Titers were reported as the concentration of mAb that inhibited 50% or 80% virus infection (IC50 and IC80 titers, respectively). All neutralization experiments were repeated twice with similar results.

### Cryo-EM Sample Preparation and Data Collection

To prepare cryo EM grids, 3.5 μl of the freshly purified sample from the peak fraction in DDM at ˜2.5 mg/ml for the Delta variant or ˜2.0 mg/ml for the Gamma and Kappa variants was applied to a 1.2/1.3 Quantifoil® grid (Quantifoil Micro Tools GmbH), which had been glow discharged with a PELCO easiGlow™ Glow™ Discharge Cleaning system (Ted Pella, Inc.) for 60 seconds at 15 mA. Grids were immediately plunge-frozen in liquid ethane using a Vitrobot Mark IV (ThermoFisher Scientific), and excess protein was blotted away by using grade 595 filter paper (Ted Pella, Inc.) with a blotting time of 4 seconds, a blotting force of −12 at 4° C. with 100% humidity. The grids were first screened for ice thickness and particle distribution. Selected grids were used to acquire images by a Titan Krios™ transmission electron microscope (ThermoFisher Scientific) operated at 300 keV and equipped with a BioQuantum™ GIF/K3™ direct electron detector. Automated data collection was carried out using SerialEM version 3.8.6 (53) at a nominal magnification of 105,000× and the K3 detector in counting mode (calibrated pixel size, 0.825 Å) at an exposure rate of 20.24 (for Delta), ˜20.69/20.63/27.13 (for three data sets of Gamma), or ˜21.12/20.10 (for two data sets of Kappa) electrons per pixel per second. Each movie add a total accumulated electron exposure of ˜51.48 (Delta), ˜54.72/54.56/53.4 (Gamma), or ˜51.63/51.15 (Kappa) e-/Å2, fractionated in 50 (Delta), 51/51/50 (Gamma), or 51/51 (Kappa) frames. Data sets were acquired using a defocus range of 0.8-2.2 (Delta), 0.8-2.3 (Gamma), or 0.8-2.2 (Kappa) μm.

### Image Processing and 3D Reconstructions

Drift correction for cryo-EM images was performed using MotionCor2 (54), and contrast transfer function (CTF) was estimated by Gctf (55) using motion-corrected sums without dose-weighting. Motion corrected sums with dose-weighting were used for all other image processing. crYOLO was used for particle picking and RELION3.0.8 for 2D classification, 3D classification and refinement procedure. For the Delta sample, 1,830,328 particles were extracted from 20,274 images using crYOLO with a trained model, and then subjected to 2D classification, giving 1,386,630 good particles. A low-resolution negative-stain reconstruction of the Wuhan-Hu-1 (D614) sample was low-pass filtered to 40 Å resolution and used as an initial model for 3D classification with C1 symmetry. After two rounds of 3D classification, two major classes with clear structural features were combined and subjected to a third round of 3D classification in C1 symmetry. The calculation led to five major classes, one in a closed, three RBD-down conformation, and four in the one-RBD-up conformation. The class of the closed conformation was re-extracted unbinned and subjected to one round of 3D auto-refinement, giving a map at 4.2 Å resolution from 125,763 particles. Additional round of signal-subtraction and 3D classification using a mask for the apex region of the S trimer were performed, leading to three distinct classes, two in the closed conformation and one in the one-RBD-up conformation. The two classes in the closed conformation were combined and subjected to another round of 3D auto-refinement, followed by CTF refinement, particle polishing, and 3D auto-refinement, producing a map at 4.2 Å resolution from 102,521 particles. Second round of signal-subtraction and 3D classification focusing on the apex region were carried out, giving one major class in the closed conformation with 94,680 particles. Further round of 3D auto-refinement in C3symmetry using a whole mask was applied to this class, followed by CTF refinement, particle polishing, and 3D auto-refinement, yielding a map at 3.1 Å resolution. Four classes in the one-RBD-up conformation from the third rounds of 3D classification were combined and particles re-extracted unbinned for one round of 3D auto-refinement, then further combined with the RBD-up class from the first round of signal-subtraction/classification based on the apex region of the closed S trimer. This combined class containing 255,909 particles was autorefined producing a map at 4.1 Å resolution. Particle CTF refinement/particle polishing and another round of autorefinement were performed before subjected to signal-subtraction and 3D classification using the apex mask. Two major classes in the one-RBD-up conformation were produced and they were subjected to 3D auto-refinement in C1symmetry using a whole mask, CTF refinement, particle polishing and a final round of 3D auto-refinement. The final reconstructions have 191,067 and 25,370 particles with maps 3.4 Å and 4.3 Å resolution, respectively. Additional rounds of 3D auto-refinement were performed for each class using different sizes of masks at the top region to improve local resolution. The best density maps were used for model building.

For the Kappa sample, two data sets were collected and initially processed separately. 1,199,999 and 1,718,600 particles were extracted using crYOLO with a trained model from 22,019 and 17,314 images, respectively, from the two sets. The selected particles were subjected to 2D classification, giving 1,112,384 and 1,649,296 particles, respectively. A low-resolution negative-stain reconstruction of the Wuhan-Hu-1 (D614) sample was low-pass-filtered to 40 Å as an initial model for 3D classification with C1 symmetry. Three rounds of 3D classification were performed separately for each data set. For data set 1, two major classes were identified, one in the closed conformation and the other in the one-RBD-up conformation. The class in the closed state was 3D auto-refined with C1 symmetry to 4.3 Å after CTF refinement and particle polishing. One round of signal-subtraction and 3D classification using the top mask was performed, giving two major classes, once again, one in the closed conformation and the other in the one-RBD-up conformation. This one-RBD-up class was combined with the RBD-up class from the previous third round of 3D classification, and refined to give a map at 4.6 Å resolution from 67,997 particles. The class in the closed state with 63,146 particles was refined to produce a map at 3.8 Å resolution after CTF refinement and particle polishing. For data set 2, 3D classification identified two classes in the closed conformation and combination of the two gave a map at 4.9 Å resolution from 171,733 particles after 3D auto-refinement. One round of signal-subtraction and 3D classification using the top mask was performed, leading to two major classes, one in the closed conformation and the other in the one-RBD-up conformation. Another round of 3D auto-refinement was carried out, giving two maps with 71,425 and 42,005 particles, respectively.

The two classes in the closed state from the two data sets were then combined and subjected to 3D auto-refinement, CTF refinement, particle polishing and a final round of 3D auto-refinement, yielding a map at 4.1 Å resolution from 255,909 particles. One round of signal-subtraction 3D and classification with the top mask was performed, leading to a major class in the closed conformation. After 3D auto-refinement with C1/C3 symmetry using a whole mask, CTF refinement, particle polishing and a final round of auto-refinement, this class yielded a final reconstruction at 3.1 Å resolution from 123,193 particles. Similarly, the one-RBD-up classes from the two date sets were combined and subjected to 3D auto-refinement, CTF refinement, particle polishing and 3D auto-refinement, producing a map at 4.0 Å resolution from 110,002 particles. One round of signal-subtraction and 3D classification using the top mask was carried out, giving two major classes in distinct one-RBD-up conformations with the RBD projecting at slightly different angles. After 3D auto-refinement with C1 symmetry using a whole mask, CTF refinement, particle polishing and a final round of auto-refinement, the two classes yielded two reconstructions at 3.7 Å and 4.3 Å resolution from 81,717 and 21,830 particles, respectively. Additional rounds of 3D auto-refinement were performed for each class using different sizes of masks at the top region to improve local resolution. The best density maps were used for model building.

For the Gamma sample, three independent data sets were collected. 1,652,420,2,757,190 and 1,893,863 particles were extracted by crYOLO with a trained model from 25,424, 32,569 and 29,163 images, respectively, from the three sets. The selected particles were subjected to 2D classification, giving 1,564,938, 1,904,078 and 1,788,000 particles, respectively. A low-resolution negative-stain reconstruction of the Wuhan-Hu-1 (D614) sample was low-pass-filtered to 40 Å as an initial model for 3D classification with C1 symmetry. Two or three rounds of 3D classification were performed separately for the three data sets and each of them produced a major class resembling an S trimer. The three classes were combined and subjected to additional round of 3D classification in C1 symmetry, leading to three good classes. The new classes were auto-refined, giving a map at 4.9 Å resolution from 143,872 particles. A round of signal-subtraction and 3D classification using the top mask were performed to produce two major classes in two distinct one-RBD-up conformations with the RBD projecting at slightly different angles. The two classes contain 69,302 and 36,346 particles, respectively and they were subjected to 3D auto-refinement with C1 symmetry using a whole mask, CTF refinement/particle polishing and a final round auto-refinement, yielding two reconstructions at 3.8 Å and 4.4 Å resolution, respectively. Additional rounds of 3D auto-refinement were performed for each class using different sizes of masks at the top region to improve local resolution. The best density maps were used for model building.

All resolutions were reported from the gold-standard Fourier shell correlation (FSC) using the 0.143 criterion. Density maps were corrected from the modulation transfer function of the K3 detector and sharpened by applying a temperature factor that was estimated using post-processing in RELION. Local resolution was determined using ResMap (56) with half-reconstructions as input maps.

### Model Building

The initial templates for model building used the G614 S trimer structures (PDB ID: 7KRQ and PDB ID: 7KRR; ref(31)). Several rounds of manual building were performed in Coot (57). The model was then refined in Phenix (58) against the 3.1 Å (closed), 3.4 Å (one-RBD-up 1), 4.3 Å (one-RBD-up 2) cryo-EM maps of the Delta variant; the 3.8 Å (one-RBD-up 1) and 4.4 Å (one-RBD-up 2) cryo-EM maps of the Gamma variant; and the 3.1 Å (closed), 3.7 Å (one-RBD-up 1) and 4.3 Å (one-RBD-up 2) cryo-EM maps of the Kappa variant. Iteratively, refinement was performed in both Refmac (real space refinement) and ISOLDE (59), and the Phenix refinement strategy included minimization_global, local_grid_search, and adp, with rotamer, Ramachandran, and reference-model restraints, using 7KRQ and 7KRR as the reference model. The refinement statistics are summarized in Table 3. Structural biology applications used in this project were compiled and configured by SBGrid (60).

Wuhan-Hu-1 (D614) Spike (S) protein
(SEQ ID NO: 2)
MFVFLVLLPLVSSQCVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFG
TTLDSKTQSLLIVNNATNVVIKVCEFQFCNDPFLGVYYHKNNKSWMESEFRV
YSSANNCTFEYVSQPFLMDLEGKQGNFKNLREFVFKNIDGYFKIYSKHTPINL
VRDLPQGFSALEPLVDLPIGINITRFQTLLALHRSYLTPGDSSSGWTAGAAAY
YVGYLQPRTFLLKYNENGTITDAVDCALDPLSETKCTLKSFTVEKGIYQTSNF
RVQPTESIVRFPNITNLCPFGEVENATRFASVYAWNRKRISNCVADYSVLYNS
ASFSTFKCYGVSPTKLNDLCFTNVYADSFVIRGDEVRQIAPGQTGKIADYNYK
LPDDFTGCVIAWNSNNLDSKVGGNYNYLYRLFRKSNLKPFERDISTEIYQAGS
TPCNGVEGENCYFPLQSYGFQPTNGVGYQPYRVVVLSFELLHAPATVCGPKK
STNLVKNKCVNFNFNGLTGTGVLTESNKKFLPFQQFGRDIADTTDAVRDPQT
LEILDITPCSFGGVSVITPGTNTSNQVAVLYQDVNCTEVPVAIHADQLTPTWR
VYSTGSNVFQTRAGCLIGAEHVNNSYECDIPIGAGICASYQTQTNSPRRARSV
ASQSIIAYTMSLGAENSVAYSNNSIAIPTNFTISVTTEILPVSMTKTSVDCTMYI
CGDSTECSNLLLQYGSFCTQLNRALTGIAVEQDKNTQEVFAQVKQIYKTPPIK
DFGGFNFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQDVVNQ
NAQALNTLVKQLSSNFGAISSVLNDILSRLDKVEAEVQIDRLITGRLQSLQTYV
TQQLIRAAEIRASANLAATKMSECVLGQSKRVDFCGKGYHLMSFPQSAPHGV
VFLHVTYVPAQEKNFTTAPAICHDGKAHFPREGVFVSNGTHWFVTQRNFYEP
QIITTDNTFVSGNCDVVIGIVNNTVYDPLQPELDSFKEELDKYFKNHTSPDVDL
GDISGINASVVNIQKEIDRLNEVAKNLNESLIDLQELGKYEQYIKWPWYIWLG
FIAGLIAIVMVTIMLCCMTSCCSCLKGCCSCGSCCKFDEDDSEPVLKGVKLHYT
G614 strain (B.1)
(SEQ ID NO: 3)
MFVFLVLLPLVSSQCVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFG
TTLDSKTQSLLIVNNATNVVIKVCEFQFCNDPFLGVYYHKNNKSWMESEFRV
YSSANNCTFEYVSQPFLMDLEGKQGNFKNLREFVFKNIDGYFKIYSKHTPINL
VRDLPQGFSALEPLVDLPIGINITRFQTLLALHRSYLTPGDSSSGWTAGAAAY
YVGYLQPRTFLLKYNENGTITDAVDCALDPLSETKCTLKSFTVEKGIYQTSNF
RVQPTESIVRFPNITNLCPFGEVENATRFASVYAWNRKRISNCVADYSVLYNS
ASFSTFKCYGVSPTKLNDLCFTNVYADSFVIRGDEVRQIAPGQTGKIADYNYK
LPDDFTGCVIAWNSNNLDSKVGGNYNYLYRLFRKSNLKPFERDISTEIYQAGS
TPCNGVEGENCYFPLQSYGFQPTNGVGYQPYRVVVLSFELLHAPATVCGPKK
STNLVKNKCVNFNFNGLTGTGVLTESNKKFLPFQQFGRDIADTTDAVRDPQT
LEILDITPCSFGGVSVITPGTNTSNQVAVLYQDVNCTEVPVAIHADQLTPTWR
VYSTGSNVFQTRAGCLIGAEHVNNSYECDIPIGAGICASYQTQTNSPRRARSV
ASQSIIAYTMSLGAENSVAYSNNSIAIPTNFTISVTTEILPVSMTKTSVDCTMYI
CGDSTECSNLLLQYGSFCTQLNRALTGIAVEQDKNTQEVFAQVKQIYKTPPIK
DFGGFNFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQDVVNQ
NAQALNTLVKQLSSNFGAISSVLNDILSRLDKVEAEVQIDRLITGRLQSLQTYV
TQQLIRAAEIRASANLAATKMSECVLGQSKRVDFCGKGYHLMSFPQSAPHGV
VFLHVTYVPAQEKNFTTAPAICHDGKAHFPREGVFVSNGTHWFVTQRNFYEP
QIITTDNTFVSGNCDVVIGIVNNTVYDPLQPELDSFKEELDKYFKNHTSPDVDL
GDISGINASVVNIQKEIDRLNEVAKNLNESLIDLQELGKYEQYIKWPWYIWLG
FIAGLIAIVMVTIMLCCMTSCCSCLKGCCSCGSCCKFDEDDSEPVLKGVKLHYT

### REFERENCES

1. P. Zhou et al., A pneumonia outbreak associated with a new coronavirus of probable bat origin. Nature 579, 270-273 (2020).
2. P. Mlcochova et al., SARS-CoV-2 B.1.617.2 Delta variant replication, sensitivity to neutralising antibodies and vaccine breakthrough. bioRxiv, 2021.2005.2008.443253 (2021).
3. U. K. G. Scientific Advisory Group for Emergencies (SAGE), SPI-M-O: Consensus Statement on COVID-19. https://assets.publishing.service.gov.uk/government/uploads/system/uploads/attachme nt_data/file/993321/S991267_SPI-M-O_Consensus_Statement.pdf (2021).
4. J. Dagpunar, Interim estimates of increased transmissibility, growth rate, and reproduction number of the Covid-19 B.1.617.2 variant of concern in the United Kingdom. medRxiv, 2021.2006.2003.21258293 (2021).
5. B. Li et al., Viral infection and transmission in a large well-traced outbreak caused by the Delta SARS-CoV-2 variant. virological.org, https://virological.org/t/viral-infection-and-transmission-in-a-large-well-traced-outbreak-caused-by-the-delta-sars-cov-2-variant/724 (2021).
6. A. Sheikh et al., SARS-CoV-2 Delta VOC in Scotland: demographics, risk of hospital admission, and vaccine effectiveness. Lancet 397, 2461-2462 (2021).
7. D. N. Fisman, A. R. Tuite, Progressive Increase in Virulence of Novel SARS-CoV-2 Variants in Ontario, Canada. medRxiv, 2021.2007.2005.21260050 (2021).
8. D. Planas et al., Reduced sensitivity of SARS-CoV-2 variant Delta to antibody neutralization. Nature 596, 276-280 (2021).
9. V. V. Edara et al., Infection and Vaccine-Induced Neutralizing-Antibody Responses to the SARS-CoV-2 B.1.617 Variants. N Engl J Med 385, 664-666 (2021).
10. J. Lopez Bernal et al., Effectiveness of Covid-19 Vaccines against the B.1.617.2 (Delta) Variant. N Engl J Med 385, 585-594 (2021).
11. T. Tada et al., Comparison of Neutralizing Antibody Titers Elicited by mRNA and Adenoviral Vector Vaccine against SARS-CoV-2 Variants. bioRxiv, 2021.2007.2019.452771 (2021).
12. N. R. Faria et al., Genomics and epidemiology of the P.1 SARS-CoV-2 lineage in Manaus, Brazil. Science 372, 815-821 (2021).
13. M. Hoffmann et al., SARS-CoV-2 variants B.1.351 and P.1 escape from neutralizing antibodies. Cell 184, 2384-2393 e2312 (2021).
14. P. D. Yadav et al., SARS CoV-2 variant B.1.617.1 is highly pathogenic in hamsters than B.1 variant. bioRxiv, 2021.2005.2005.442760 (2021).
15. V.-V. Edara et al., Infection and vaccine-induced neutralizing antibody responses to the SARS-CoV-2 B.1.617.1 variant. bioRxiv, 2021.2005.2009.443299 (2021).
16. B. J. Bosch, R. van der Zee, C. A. de Haan, P. J. Rottier, The coronavirus spike protein is a class I virus fusion protein: structural and functional characterization of the fusion core complex. J Virol 77, 8801-8811 (2003).
17. M. Hoffmann et al., SARS-CoV-2 Cell Entry Depends on ACE2 and TMPRSS2 and Is Blocked by a Clinically Proven Protease Inhibitor. Cell 181, 271-280 e278 (2020).
18. J. K. Millet, G. R. Whittaker, Host cell entry of Middle East respiratory syndrome coronavirus after two-step, furin-mediated activation of the spike protein. Proc Natl Acad Sci U S A 111, 15214-15219 (2014).
19. M. A. Tortorici, D. Veesler, Structural insights into coronavirus entry. Adv Virus Res 105, 93-116 (2019).
20. D. Wrapp et al., Cryo-EM structure of the 2019-nCOV spike in the prefusion conformation. Science 367, 1260-1263 (2020).
21. J. Shang et al., Cell entry mechanisms of SARS-CoV-2. Proc Natl Acad Sci U S A 117, 11727-11734 (2020).
22. Y. Huang, C. Yang, X. F. Xu, W. Xu, S. W. Liu, Structural and functional properties of SARS-CoV-2 spike protein: potential antivirus drug development for COVID-19. Acta Pharmacol Sin 41, 1141-1149 (2020).
23. C. Zhu et al., Molecular biology of the SARs-CoV-2 spike protein: A review of current knowledge. J Med Virol 93, 5729-5741 (2021).
24. N. Murgolo et al., SARS-CoV-2 tropism, entry, replication, and propagation: Considerations for drug discovery and development. PLOS Pathog 17, e1009225 (2021).
25. R. Peng, L. A. Wu, Q. Wang, J. Qi, G. F. Gao, Cell entry by SARS-CoV-2. Trends Biochem Sci, (2021).
26. Y. Cai et al., Structural basis for enhanced infectivity and immune evasion of SARS-CoV-2 variants. Science 373, 642-648 (2021).
27. B. Korber et al., Tracking Changes in SARS-CoV-2 Spike: Evidence that D614G Increases Infectivity of the COVID-19 Virus. Cell 182, 812-827 e819 (2020). 28. Y. Cai et al., Distinct conformational states of SARS-CoV-2 spike protein. Science 369, 1586-1592 (2020).
29. J. Yu et al., Deletion of the SARS-CoV-2 Spike Cytoplasmic Tail Increases Infectivity in Pseudovirus Neutralization Assays. J Virol, (2021).
30. T. Giroglou et al., Retroviral vectors pseudotyped with severe acute respiratory syndrome coronavirus S protein. J Virol 78, 9007-9015 (2004).
31. J. Zhang et al., Structural impact on SARS-CoV-2 spike protein by D614G substitution. Science 372, 525-530 (2021).
32. P. Tong et al., Memory B cell repertoire for recognition of evolving SARS-CoV-2 spike. Cell, (2021).
33. T. Xiao et al., A trimeric human angiotensin-converting enzyme 2 as an anti-SARS-CoV-2 agent. Nat Struct Mol Biol 28, 202-209 (2021).
34. T. Wagner et al., SPHIRE-crYOLO is a fast and accurate fully automated particle picker for cryo-EM. Commun Biol 2, 218 (2019).
35. S. H. Scheres, RELION: implementation of a Bayesian approach to cryo-EM structure determination. J Struct Biol 180, 519-530 (2012).
36. J. Lan et al., Structure of the SARS-CoV-2 spike receptor-binding domain bound to the ACE2 receptor. Nature 581, 215-220 (2020).
37. J. Shang et al., Structural basis of receptor recognition by SARS-CoV-2. Nature 581, 221-224 (2020).
38. A. C. Walls et al., Structure, Function, and Antigenicity of the SARS-CoV-2 Spike Glycoprotein. Cell 183, 1735 (2020).
39. S. M. Gobeil et al., Effect of natural mutations of SARS-CoV-2 on spike structure, conformation, and antigenicity. Science 373, (2021).
40. E. Volz et al., Assessing transmissibility of SARS-CoV-2 lineage B.1.1.7 in England. Nature 593, 266-269 (2021).
41. M. U. G. Kraemer et al., Spatiotemporal invasion dynamics of SARS-CoV-2 lineage B.1.1.7 emergence. Science, (2021).
42. N. G. Davies et al., Estimated transmissibility and impact of SARS-CoV-2 lineage B.1.1.7 in England. Science 372, (2021).
43. S. Kim et al., Differential Interactions Between Human ACE2 and Spike RBD of SARS-CoV-2 Variants of Concern. bioRxiv, 2021.2007.2023.453598 (2021).
44. A. Saito et al., SARS-CoV-2 spike P681R mutation enhances and accelerates viral fusion. bioRxiv, 2021.2006.2017.448820 (2021).
45. Y. Liu et al., Delta spike P681R mutation enhances SARS-CoV-2 fitness over Alpha variant. bioRxiv, 2021.2008.2012.456173 (2021).
46. X. Chi et al., A neutralizing human antibody binds to the N-terminal domain of the Spike protein of SARS-CoV-2. Science 369, 650-655 (2020).
47. L. Liu et al., Potent neutralizing antibodies against multiple epitopes on SARS-CoV-2 spike. Nature 584, 450-456 (2020).
48. C. O. Barnes et al., Structures of Human Antibodies Bound to SARS-CoV-2 Spike Reveal Common Epitopes and Recurrent Features of Antibodies. Cell 182, 828-842 e816 (2020).
49. P. D. Yadav et al., Comparable neutralization of SARS-CoV-2 Delta AY.1 and Delta in individuals sera vaccinated with BBV152. bioRxiv, 2021.2007.2030.454511 (2021).
50. J. Chen et al., HIV-1 ENVELOPE. Effect of the cytoplasmic domain on antigenic characteristics of HIV-1 envelope glycoprotein. Science 349, 191-195 (2015).
51. C. Z. Chen et al., Identifying SARS-CoV-2 Entry Inhibitors through Drug Repurposing Screens of SARS-S and MERS-S Pseudotyped Particles. ACS Pharmacol Transl Sci 3, 1165-1175 (2020).
52. J. K. Millet, G. R. Whittaker, Murine Leukemia Virus (MLV)-based Coronavirus Spike-pseudotyped Particle Production and Infection. Bio Protoc 6, (2016).
53. D. N. Mastronarde, Automated electron microscope tomography using robust prediction of specimen movements. J Struct Biol 152, 36-51 (2005).
54. S. Q. Zheng et al., MotionCor2: anisotropic correction of beam-induced motion for improved cryo-electron microscopy. Nat Methods 14, 331-332 (2017).
55. K. Zhang, Gctf: Real-time CTF determination and correction. J Struct Biol 193, 1-12 (2016).
56. A. Kucukelbir, F. J. Sigworth, H. D. Tagare, Quantifying the local resolution of cryo-EM density maps. Nat Methods 11, 63-65 (2014).
57. P. Emsley, B. Lohkamp, W. G. Scott, K. Cowtan, Features and development of Coot. Acta Crystallogr D Biol Crystallogr 66, 486-501 (2010).
58. P. D. Adams et al., PHENIX: a comprehensive Python-based system for macromolecular structure solution. Acta Crystallogr D Biol Crystallogr 66, 213-221 (2010).
59. T. I. Croll, ISOLDE: a physically realistic environment for model building into low-resolution electron-density maps. Acta Crystallogr D Struct Biol 74, 519-530 (2018).
60. A. Morin et al., Collaboration gets the most out of software. Elife 2, e01456 (2013).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

What is claimed is:

1. A method for identifying a therapeutic for the treatment of SARS-CoV-2, said method utilizing the receptor-binidng domain (RBD) of the SARS-CoV-2 S protein.

2. The method of claim 1, wherein the therapeutic is a small molecule.

3. The method of claim 1, wherein the therapeutic is a biologic.

4. The method of claim 1, wherein the therapeutic is a protein.

5. The method of claim 1, wherein the therapeutic is an antibody.

6. The method of any one of claim 1-5, wherein the therapeutic recognizes or binds to the RBD-1 region of the RBD domain.

7. The method of any one of claim 1-5, wherein the therapeutic recognizes or binds to the RBD-2 region of the RBD domain.

8. The method of any one of claim 1-5, wherein the therapeutic recognizes or binds to the RBD-3 region of the RBD domain.

9. A method of treating a patient suffering from SARS-CoV-2, wherein the method comprises the step of administering to the patient a therapeutically effective amount of the therapeutic identified in any of claims 1-9.

10. A method for developing a vaccine for the treatment or prevention of SARS-CoV-2, said method utilizing the receptor-binidng domain (RBD) of the SARS-CoV-2 S protein.

11. The method of claim 10, wherein the RBD-1 region of the RBD domain is utilized.

12. The method of claim 10, wherein the RBD-2 region of the RBD domain is utilized.

13. The method of claim 10, wherein the RBD-3 region of the RBD domain is utilized.

14. A method for administering to a subject a vaccine for the treatment of SARS-CoV-2, wherein the method comprises the step of administering to the subject an immunogenically effective amount of the vaccine identified in any of claims 10-13.

15. A method for identifying a patient as being in need for a treatment for SARS-CoV-2, said method utilizing the receptor-binidng domain (RBD) of the SARS-CoV-2 S protein.

16. The method of claim 10, wherein the RBD-1 region of the RBD domain is utilized.

17. The method of claim 11, wherein the RBD-2 region of the RBD domain is utilized.

18. The method of claim 11, wherein the RBD-3 region of the RBD domain is utilized.

19. A method of treating a patient identified in any one of claims 15-18, wherein the method comprises the step of administering a thearputic for the treatment of SARS-CoV-2.

20. A method of treating a patient identified in any one of claims 15-18, wherein the method comprises the step of administering a vaccine directed to SARS-CoV-2.
